(19) United States
(12) Patent Application Publication
(10) Pub. No.: US 2026/0360480 A1
(43) Pub. Date: Nov. 12, 2026

(54) SYSTEM AND METHOD HAVING MOLECULAR REPRESENTATION FOR DEEP LEARNING OF MOLECULAR PROPERTY DESIGN AND PREDICTION

(71) Applicant: Purdue Research Foundation, West Lafayette, IN (US)

(72) Inventor: Tonglei Li, West Lafayette, IN (US)

(73) Assignee: Purdue Research Foundation, West Lafayette, IN (US)

(21) Appl. No.: 19/121,658

(22) PCT Filed: Oct. 13, 2023
(86) PCT No.: PCT/US2023/035079

§ 371 Date: Apr. 16, 2025

Related U.S. Application Data
(60) Provisional application No. 63/416,723, filed on Oct. 17, 2022.

(51) Int. Cl. G16C 20/50; G16C 20/30; G16C 20/70

(52) U.S. Cl. CPC G16C 20/50; G16C 20/30; G16C 20/70

(57) ABSTRACT

The present invention involves a method, a system, and software for a method of generating unique descriptors of intermolecular interactions by: 1) using quantum mechanical conceptual density functional theory (CDFT) calculations to generate measures of electron density from crystal structures; 2) generating the molecular surface by determining the contribution of electron in the crystal structure from individual molecules; and 3) creating a method called Manifold Embedding of the Molecular Surface (MEMS). MEMS embeds the CDFT calculated values onto the three-dimensional (3D) molecular surface which is then projected into two dimensions (2D) using a stochastic neighborhood embedding approach. From each atom location in the 2D space, radially and angularly distributed electrostatic potential and Fukui function values are taken as input for the neural network.

### CROSS-REFERENCE TO RELATED APPLICATIONS

The present application is an International Patent application claiming priority from the U.S. provisional patent application Ser. No. 63/416,723, filed on Oct. 17, 2022, the disclosures of which are incorporated by reference in their entireties.

### BACKGROUND OF THE INVENTION

### Field of the Invention

The invention relates to data analytics. More specifically, the present disclosure relates to computational methods, systems, devices and/or apparatuses for molecular representation for deep learning of molecular properties prediction and de novo design.

### Description of the Related Art

Drug discovery and development is often described as finding a needle in a haystack. In fact, searching for a drug in the chemical space (10\hat{ }63 molecules and more) is much more daunting, especially when dozens or more molecular descriptors are used to define the dimensions of the space. Identifying and optimizing a fitting chemical through the drug discovery and development processes is costly and laborious. As a result, less than 1 out of 10\hat{ }4 molecules brought to the drug research pipeline can make it to the clinic. The extreme complexities in evaluating a lead compound's efficacy, toxicity, bioavailability, and developability have long desired in silico approaches to predict the physical, biological, and pharmacological properties of molecules based on their structures. Recent advances in deep learning and the availability of extensive chemical data provide great opportunities to facilitate drug research; however, prediction of molecular functionalities and de novo drug design has not yet made great strides. A root obstacle is the Curse of Dimensionality (COD) when exploring the chemical space by current schemes of molecular representation.

A molecule is classically depicted by a graph of nodes and lines. Various featurization schemes have been empirically evolved from the graphic convention. When predicting molecular interactions and properties, it is a common practice to use as many descriptors (and fingerprints) as possible to embody the chemistry of a molecule. However, the more features are used, the more exponentially the dimensionality of the chemical space expands. The COD leaves the vast space scarcely covered by available experimental data, drastically deteriorating the predictive power of data-fitted structure-property models.

In silico drug research counts on computable features of molecules to exploit available chemical data and explore the underlying quantitative relationships between molecular features and conceivable physicochemical and therapeutic properties. Thanks to the rapid growth in data collection, cloud storage, and online access, various datacentric approaches have become a staple in modern drug discovery and development. At the same time, prediction by first principles remains impractical, especially when dealing with multifaceted phenomena (e.g., dissolution and binding to a flexible protein). Most featurization schemes are empirically evolved from the conventional depiction of a molecule as a graph of atoms, resulting in many forms of descriptors and fingerprints. A descriptor is a value of a molecule's 1-, 2-, or 3-D feature (e.g., number of hydrogen-bonding donors). A fingerprint consists of an alphanumerical string (e.g., Weininger, D., Smiles, “A Chemical Language and Information-System.1. Introduction to Methodology and Encoding Rules” [Journal of Chemical Information and Computer Sciences 1988, 28, 31-36]) or a digital vector (e.g., Morgan, H. L., “Generation of a Unique Machine Description for Chemical Structures-a Technique Developed” [at Chemical Abstracts Service, Journal of Chemical Documentation 1965, 5, 107-&]) and (Rogers, D.; Hahn, M., “Extended-Connectivity Fingerprints” [Journal of Chemical Information and Modeling 2010, 50, 742-754]) to encode the holistic chemical constitution and bonding information. However, no single descriptor or fingerprint can fully capture the chemistry of a molecule's functionality. It is thus not uncommon to see in a study that dozens or even hundreds of features are used to represent a molecule. The practice, unfortunately, leads to COD. Considering the sheer number of potential molecule candidates (>10\hat{ }63)19, the Curse is additionally exacerbated.

The chemical space formed by conventional descriptors (and fingerprints) becomes too sparse to cover by chemical data, resulting in model overfitting. The predictive power of any data-derived model exponentially deteriorates as the number of descriptor dimensions increases. The COD has primarily impeded the data-driven drug discovery and development, making it critically desired to create low-dimensional molecular features that accurately capture the chemistry of a molecule.

Earlier work has provided some attempts to improve the computation of the chemical space, see, e.g., Li, T. L.; Liu, S. B.; Feng, S. X.; Aubrey, C. E., “Face-Integrated Fukui Function: Understanding Wettability Anisotropy of Molecular Crystals from Density Functional Theory” (Journal of the American Chemical Society 2005, 127, 1364-1365); Zhang, M. T.; Li, T. L., “Intermolecular Interactions in Organic Crystals: Gaining Insight from Electronic Structure Analysis by Density Functional Theory” (Crystengcomm 2014, 16, 7162-7171); Bhattacharjee, R.; Verma, K.; Zhang, M.; Li, T. L., “Locality and Strength of Intermolecular Interactions in Organic Crystals: Using Conceptual Density Functional Theory (Cdft) to Characterize a Highly Polymorphic System” (Theoretical Chemistry Accounts 2019, 138).

### SUMMARY OF THE INVENTION

The present invention involves, in one embodiment, mapping molecules to a low-dimensional, quantum chemical space so that drug candidates that achieves desired therapeutic and bioavailable properties will be effectively identified with deep learning of chemical data.

Several embodiments of the invention involve transforming a molecule's 3-D electronic attributes of local hardness and softness to a 2-D manifold embedding. Such a representation and resulting data structure carries the inherent information of intermolecular interaction strength and specificity, circumventing the COD by being further featurized and orthogonally reduced to a low-dimensional latent space of quantum chemistry, thus, building a deep-learning framework around the reduced latent space to predict molecular properties and search for new molecules to treat various diseases. The improved data structure (1) to model such small molecules and utilize their models in deep learning to predict essential bioavailable properties of small molecules; (2) to apply the bioavailable properties of those models in deep learning models to virtually screen small molecules for a few selected diseases (including without limitation Malaria, COVID-19, and HIV; and (3) to provide deep learning models of de novo drug design for the studied diseases in reference to the optimal targeting and bioavailable properties predicted in the preceding aims. The inventive data structure and model transforms the broad area of chemical learning from an art of describing a molecule, which is however shadowed by COD, to a closed-form solution that is vigorously defined by quantum mechanics and manifold learning. Promising compounds generated by the de novo design may be tested in separate studies. With the provision of online tools and datasets of calculated molecular features, small molecule research and development may be greatly enhanced.

Manifold Embedding of Molecular Surface (MEMS) is an aspect of the present invention wherein data structures and models, instead of describing a molecule's atomic and bonding information, are represented by lower-dimensional embeddings of the electronic density and pertinent attributes on a molecular surface. By progressively reducing the dimensionalities of MEMS, one may project a molecule in a lowdimensional space of quantum chemistry. As schematically shown in FIG. 1, embodiments of the invention involve transforming quantum chemical quantities on a molecular surface to low-dimensional latent variables by multiple dimensionality reduction steps, including Stochastic Neighborhood Embedding (SNE), Shape Context (SC), Gaussian Process (GP), and GP Latent Variable Model (GPLVM). The significance of MEMS lies in systemically unifying molecules in a tractable and chemically differentiating space to enable the data-driven discovery of mutual connections between molecular structures and functionalities. MEMS and latent projections essentially preserve the inherent chemical information of molecular interactions. MEMS is innovative as it utilizes the local electronic attributes on a molecular surface and identifies their lower-dimensional embeddings as the sole feature of the molecule. Preliminary results of solubility prediction and CPY450 binding show great promises of using MEMS in deep learning. By building a deep learning platform around the MEMS data structures and modeling to advance data-driven property predictions and molecule and product designs in broad areas of human disease.

The model of embodiments of the invention involves a Deep Sets-based Graph and Self-Attention Network with input from a computational chemistry approach to evaluating electron density and solubility values as labels. As intermolecular interactions are very important in determining the solubility of a molecule and are represented well by electron density, electron density of the drug molecules may be used computationally to predict solubility. A good dataset is critical to the success of a solubility prediction algorithm. In one embodiment, the algorithm uses molecules found in the first and second Solubility Challenge conducted by Avdeef et. al, a solubility prediction challenge which pitted human predictors against machine learning algorithms. There are a total of 90 molecules with an inherently normal distribution of solubility values. The method of generating unique descriptors of intermolecular interactions was accomplished by: 1) using quantum mechanical conceptual density functional theory (CDFT) calculations to generate measures of electron density from crystal structures; 2) generating the molecular surface by determining the contribution of electron in the crystal structure from individual molecules; and 3) creating a method called Manifold Embedding of the Molecular Surface (MEMS). MEMS embeds the CDFT calculated values onto the three-dimensional (3D) molecular surface which is then projected into two dimensions (2D) using a stochastic neighborhood embedding approach. From each atom location in the 2D space, radially and angularly distributed electrostatic potential and Fukui function values are taken as input for the neural network.

### BRIEF DESCRIPTION OF THE DRAWINGS

The above mentioned and other features and objects of this invention, and the manner of attaining them, will become more apparent and the invention itself will be better understood by reference to the following description of an embodiment of the invention taken in conjunction with the accompanying drawings, wherein:

FIG. 1 is a schematic diagrammatic flow diagram of the MEMS system according to one embodiment of the present invention.

FIG. 2 is high level process flow diagram according to several embodiments of the present invention.

FIG. 3 shows molecular mappings using various mapping methods.

FIG. 4 shows a progression of dimensionality reduction in several images of tolfenamic acid.

FIG. 5 shows MEMS illustrations of polymorphs of tolfenamic acid.

FIG. 6 shows the Shape Context transformation of one embodiment of the present invention.

FIGS. 7A and 7B are graphical results of training accuracy according to one embodiment of the present invention.

FIGS. 8A and 8B show MEMS representations generated by RBF interpolation and SGP, respectively.

FIG. 9 is a series of graphs showing MEMS (shape context) of 162 latent molecules reduced to 3-D latent spaces by GPLVM.

FIG. 10 is a picture illustrating surface points on a MEMS representation.

FIG. 11 are pictures showing a RBF interpolation filter.

FIG. 12 are illustrations of dimensionality reduction of manifolds.

FIG. 13 provides both an illustration of tolfenamic acid conformers and MEMS representations.

FIG. 14 is a schematic process diagram one embodiment of the computational prediction system and method of the present invention.

FIG. 15 is a schematic process diagram one embodiment of the computational de novo development system and method of the present invention.

FIG. 16 is a schematic diagrammatic view of a network system in which embodiments of the present invention may be utilized.

FIG. 17 is a block diagram of a computing system (either a server or client, or both, as appropriate), with optional input devices (e.g., keyboard, mouse, touch screen, etc.) and output devices, hardware, network connections, one or more processors, and memory/storage for data and modules, etc. which may be utilized in conjunction with embodiments of the present invention.

Corresponding reference characters indicate corresponding parts throughout the several views. Although the drawings represent embodiments of the present invention, the drawings are not necessarily to scale and certain features may be exaggerated in order to better illustrate and explain the present invention. The flow charts and screen shots are also representative in nature, and actual embodiments of the invention may include further features or steps not shown in the drawings. The exemplification set out herein illustrates an embodiment of the invention, in one form, and such exemplifications are not to be construed as limiting the scope of the invention in any manner.

### DESCRIPTION OF EMBODIMENTS OF THE PRESENT INVENTION

The embodiment disclosed below is not intended to be exhaustive or limit the invention to the precise form disclosed in the following detailed description. Rather, the embodiment is chosen and described so that others skilled in the art may utilize its teachings.

In the field of molecular modeling, Manifold Embedding of Molecular Surface (MEMS) is aimed to represent a molecule as a chemically authentic and dimensionally reduced feature for computing molecular interactions and pertinent properties. It aligns with Manifold Learning by treating a molecular surface as a manifold and seeking its lower-dimensional embedding through dimensionality reduction. A molecular surface marks the boundary where intermolecular interactions-attraction and repulsion-mostly converge. It is well established that electronic attributes on a molecular surface, including electrostatic potential (ESP) and Fukui functions, determine both the strength and locality of intermolecular interactions. By preserving the spatial distribution of electronic quantities on a molecular surface to its 2-D embedding(s), MEMS represents a molecule with respect to its inherent chemistry of molecular interactions. Importantly, the underlying dimensionality of the electronic patterns on a molecular surface and its embedding is much smaller than that of MEMS. As the electronic structure and properties of a molecule are determined by its atoms and their relative positions, the true dimensionality of MEMS is similarly defined and may be uncovered by Shape Context (SC) or Gaussian Process (GP). In some embodiments, the derived GP parameters may be used to represent MEMS for deep learning. The dimensionality of MEMS features (by SC or GP) may be further reduced (e.g., Lawrence, N., “Probabilistic Non-Linear Principal Component Analysis with Gaussian Process Latent Variable Models” [Journal of Machine Learning Research 2005, 6, 1783-1816] or Kingma, D. P.; Welling, M., “Auto-Encoding Variational Bayes” [arXiv Preprint 2013, arXiv: 1312.6114]) to defeat the COD.

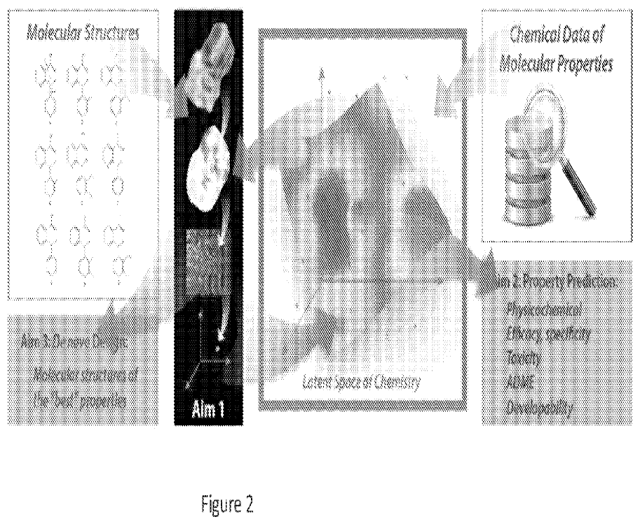

The significance of MEMS and its potential impact arises from the originality to featurize a molecule. By quantum mechanically “digesting” molecular structures, refining, and unifying the obtained information as manifold embeddings, MEMS consistently represents every molecule by preserving both the numerical values and spatial relations of the local electronic attributes on its surface. As it is mathematically derived from the local electronic properties of a molecule, MEMS may solely institute a unified quantum chemical space to potentially differentiate all molecules. As shown in FIG. 2, which shows a flow process which systemically unifying molecules in a low-dimensional, discriminative space of quantum chemistry by MEMS for the deep learning of molecular properties and de novo design of molecules and drug products the low-dimensional latent space, compared to that formed by conventional descriptors (and fingerprints), which may effectively and mutually bridge molecular structures and chemical properties. Sampling the space by respective datasets could uncover the underlying relationships of efficacy, toxicity, ADME (absorption, distribution, metabolism, and excretion) functionality, and product developability (solubility, physicochemical stability, mechanical strength, etc.). Importantly, having a structure-property relationship smoothly established in the latent space enables the optimization and discovery of the “best” MEMS of the property of interest. By reserving the manifold embedding process through generative deep learning, we may subsequently “recover” molecular structures of any given MEMS. A huge amount of chemical data has been generated to date and is expected to accumulate at an accelerated pace, all of which may be converted to MEMS. Concurrently, deep learning has rapidly grown in recent years thanks to the exponential advance in computing hardware software and the demonstrated capabilities in potentially approximating any latent functions. By reducing electronic attributes on a molecular surface to 2-D embeddings as matrices, the power of tensor and GPU computing may be unleashed for chemical deep learning. The 2-D format also makes it straightforward to stack additional layers of information (such as MEMS of the conformers of a molecule). Embodiments of MEMS significantly advance datacentric drug research and fully utilize various data collections in drug discovery and development. By breaking COD, researchers in many areas may be unbridled to freely explore the chemical space and search for cures for diseases.

A molecular surface with electronic density and pertinent attributes is widely adopted in chemical studies to gain insights into molecular interactions, reaction mechanisms, and various physicochemical phenomena. However, it has never been attempted before to treat a molecular surface as a manifold, compute its lower-dimensional embedding(s), and preserve the electronic properties of the manifold embedding. MEMS provides a data structure and model of molecular representation for chemical learning to predict molecular interactions and physicochemical, biological, and pharmacological properties. The inspiration was borne out of our earlier studies of developing electron density-based quantities to characterize the locality and strength of intermolecular interactions. As the inventor attempted to match the local electronic quantities between two molecular surfaces and calculate the interaction strength, dimensionality reduction of molecular surfaces thus became a viable direction of pursuit with manifold learning and implemented a method of manifold embedding. Preliminary results reassure the groundwork that makes the MEMS concept truly distinctive: preserving the quantum chemical attributes on a molecular surface by a lower-dimensional embedding.

Because a molecular surface is enclosed, some surface points would fall in a “wrong” neighborhood when the manifold is reduced to a 2-D embedding. Cutting open a line on a surface can minimize the falsehood, except for points along the cutting line. The totality of information may be perceived by linearly combining the embeddings of several cuts as a final representation of the molecule. Similarly, linearly stacking the MEMS of a molecule's conformers (weighed by the respective conformational energies) carries additional information on the interaction specificity of the molecule when binding with flexible (or unknown) targets. By using manifold cutting and integrating the MEMS of conformers, we could strengthen the originality and broaden the applicability of the MEMS concept. Eventually, calculated MEMS libraries of molecules under various chemical environments may be provided to end users to utilize in their drug research.

More importantly, the low-dimensional features extracted from MEMS by SC and GP are further mapped to a lower-dimensional latent space by Gaussian Process Latent Variable Models (GPLVM) and Variation AutoEncoders (VAE). The latent space is essentially formed by quantum chemical dimensions that could serve as a singular universe to host every molecule discriminatively. As the mapping of a molecule to the latent space is achieved by considering probability distributions of MEMS features (via Bayes' Theorem), a functional surface could be truthfully (and smoothly) estimated or learned by using chemical data of the function (or property) of interest. The smooth function with the variance information could facilitate generative modeling of MEMS based on a molecular property, e.g., by Born-Oppenheimer approximation (BO). To reversely project a given MEMS to its causal molecular structures will be achieved by deep learning, in which the surface electronic features are connected to the covalent information of a molecule. The efforts create an entirely new avenue for the de novo design of molecules from the root of intermolecular interactions. Moreover, it is intriguing and potentially rewarding to investigate deterministic linkages between the GP (of a particular electronic attribute) on a molecular surface and the Gaussian-based atomic orbitals of the molecule. Such connection may advance chemical deep learning to a higher level, e.g., by directly approximating latent functions between molecular properties and the structure of a molecule without resorting to MEMS. Additionally, MEMS may become a holistic molecule representation widely adopted by in silico drug research. Because of its tensor format, MEMS is readily processed by computers. MEMS mitigates the loss of quantum chemical information of manifold embedding, integrates the information of conformational space of a molecule, further reduces the dimensionality of MEMS, and may develop further generative models of MEMS and molecular structures.

Drug discovery and development essentially revolves around assessing intermolecular interactions manifested as efficacy, toxicity, bioavailability, and developability. Molecular surfaces bearing electronic attributes, such as electrostatic potential (ESP), are often used to understand the strength of molecular interactions and, more importantly, the specificity or regioselectivity that are determined by local electronic structures and attributes, as well as by the spacing and alignment between the interacting molecules. Over the last several decades, the electronic attributes developed out of the Conceptual Density Functional Theory (CDFT) have proved insightful and predictive of reaction mechanisms and molecular interactions. Several essential attributes, including the Fukui function, are intimately connected with the Hard and Soft Acids and Bases Principle (HSAB). Being a local electronic perturbation-response quantity, Fukui function is directly proportional to the local softness or polarizability of a molecular system. It is defined as the partial derivative of the local electron density with respect to the number of electrons (N). Because of the discontinuity of N, Fukui function is further defined as nucleophilic (f+, due to increase in N) and electrophilic (f−, due to decrease in N) functions; the difference (f+−f−) is dual descriptor (f2). An outstanding region of Fukui function contributes considerably to the local and overall non-covalent interactions. Similarly, while an unambiguous solution is lacking for local hardness, ESP has been used for examining hard-hard interactions because it is capable of probing the local hardness. The inventor has exploited the local HSAB and developed several CDFT concepts to characterize the locality and strength of intermolecular interactions in organic crystals. The findings unveil that Fukui functions and electrostatic potential (ESP) quantitatively determine the locality and strength of intermolecular interactions, when examined at the interface between two molecules. In an organic crystal, such an interacting interface may be epitomized by Hirshfeld surface. One finding was that the electronic properties of the single molecule of interest—other than those of the explicitly interacted molecule pair—determine both the strength and locality of intermolecular interactions to be formed. That finding implies that the intrinsic electronic structure and local electronic attributes of an isolated molecule carry the inherent information about how the molecule interacts. Therefore, embodiments of the invention develop and apply CDFT concepts in drug research, especially the prediction of supramolecular packing and assembly and binding of small molecules with proteins.

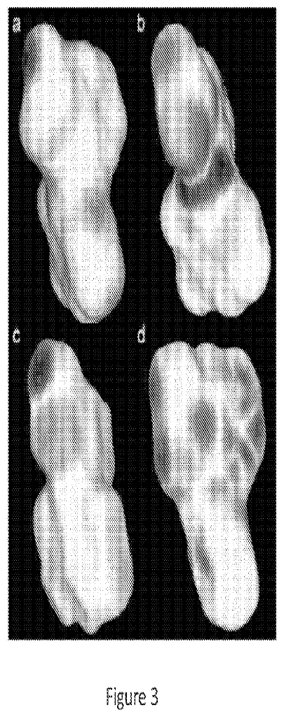

Several molecular surfaces are mapped by ESP and Fukui functions (f2) from our studies are shown in FIG. 3, where solvent-exclusion surface (a & b) and Hirshfeld surface (c & d) of tolfenamic acid mapped with ESP (a & c) and f 2 (b & d) are shown. The electronic patterns visibly indicate local electronic attributes of hardness and softness. A particular intermolecular interaction (e.g., hydrogen bonding or π-π stacking) is governed by the innate attributes at the contact area between the two molecules. We thus tried to match the local hardness and softness of molecules to predict crystal packing and related physical properties. We were also tempted to understand how a ligand molecule would fit into a protein pocket based on matching local electronic attributes between the ligand and protein surface. Nonetheless, we would run into the COD for either case as there are at least six degrees of freedom to arrange two molecules in space. This frustration has led to MEMS.

In addition, as the local electronic properties decide the interaction strength between two molecules, calculating the interaction energy directly from the local electronic values on the molecular surface(s) becomes advantageous. The challenge to identify feasible theories and mathematical functions led to our full embrace of neural networks. According to the Universal Approximation Theorem, any function could be approximated by neural networks. By developing a suitable network architecture and training it with data, the unknown function may be uncovered by approximation.

Treating a molecular surface as a manifold (specifically, Riemannian manifold), our MEMS concept roots in Manifold Learning. To generate manifold embeddings, we have implemented a non-linear method of Stochastic Neighbor Embedding (SNE), Neighbor Retrieval Visualizer (NeRV). The process preserves the local neighborhood of surface points between the manifold and embedding. The neighborhood is defined by pairwise geodesic distances among surface vertices of the manifold mesh (e.g., Hirshfeld surface or solvent-exclusion surface). The neighborhood is evaluated as the probability of vertex j in the neighborhood of vertex i:

$${p}_{j \vert i} = \frac{exp ( - {d}_{ij}^{2} / {σ}_{i}^{2} )}{{\sum }_{k \neq i} exp ( - {d}_{ik}^{2} / {σ}_{i}^{2} )} \qquad ( Eq . 1 )$$

where dᵢⱼ is the geodesic distance and σᵢ is a predefined hyperparameter of neighborhood coverage. A similar probability is defined by the Euclidean distance between the points i and j on the lower-dimensional embedding. Kullback-Leibler (KL) divergence is used as the cost function to optimize the latter probability distribution. Electronic properties on the molecule surface are pointwisely mapped to the MEMS.

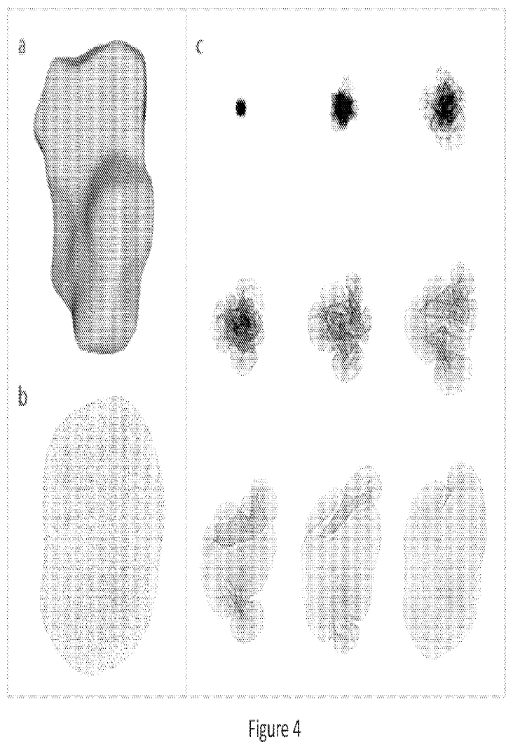

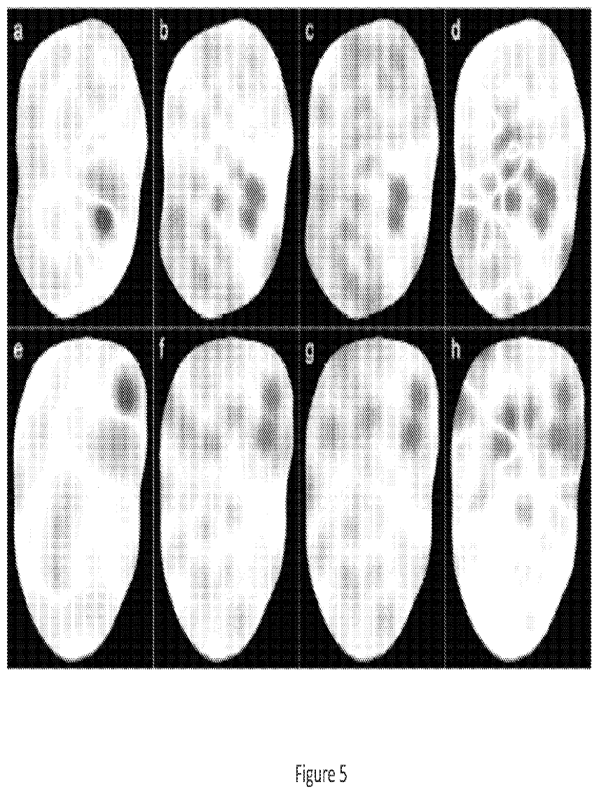

The dimensionality reduction process of a Hirshfeld surface of tolfenamic acid (metastable or Form II) is illustrated in FIG. 4, where dimensionality reduction of the Hirshfeld surface of tolfenamic acid (a) to its 2-D embedding (b) are shown as well as selected immediate steps of the KL optimization in (c). The surface was generated by Tonto, and the vertices were further optimized by isotropic remeshing in MeshLab56 (4a). Finally, the mesh vertices were input to our C++ program of KL optimization, producing 2-D points of MEMS (4b). The optimization process is demonstrated in 4c, where the initially randomized points were progressively repositioned, finally reaching a local minimum of the cost function. FIG. 5 shows the interpolated MEMS color-coded with electronic properties on the corresponding manifolds. The electronic properties were calculated by Gaussian 09 of the respective single molecule, whose conformation was extracted from the crystal structure of Form I or II. The interpolation of electronic values was conducted by Gaussian-based radial basis functions (RBFs). Because RBF is a smooth function, it preserves dominant electronic attributes and smooths out minor features (false positives and negatives) on MEMS. The MEMS in FIG. 5 are of the same molecule but of different conformers, where dominant electronic properties and their spatial patterns seem to be preserved, more particularly where MEMS of two polymorphs of tolfenamic acid, Form II (a-d) and Form I (e-h) are shown having the electronic properties from left to right of ESP, f⁺, f¹, and f². The conformational difference is also truthfully reflected in the MEMS. Note that the color scale in the MEMS in FIG. 5 is relative to the respective electronic attributes. Each image has its largest absolute value scaled to the full byte with positive numbers assigned to the red and negative to the blue channel, except for ESP where the red and blue are switched.

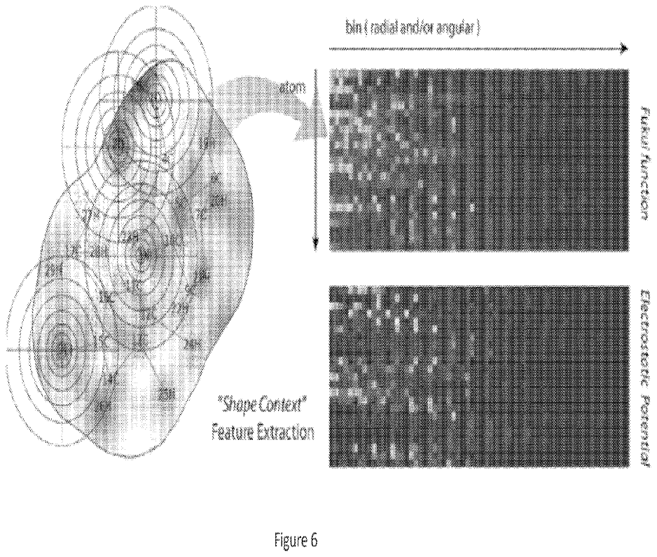

The intricacy of electronic attributes on a MEMS provides advantages to predict molecular interactions is by deep learning. It is possible to directly feed MEMS into the computer as an image and utilize CNN (convolutional neural network) for learning. Yet, the electronic pattern on a MEMS is relatively simple compared with real-life images typically used in CNN, and seemingly comprised of overlapping 2-D bell-shaped functions centering around a few surface points. Embodiments of the invention provide a featurization method based on Shape Context in computer vision. Show in FIG. 6, where the shape contexts on four atoms are illustrated with the feature matrices are of the same molecule but different electronic properties, an SC feature matrix consists of rows of key points, which are the closest surface vertices to the respective atoms of the molecule in 3-D (denoted by atom indices on the figure). The intensities surrounding a key point on a MEMS image are spatially separated in predetermined bins along the radial direction. Each radial bin may be further divided into angular bins, where the angular direction is calculated against the geometric center to allow the rotational invariance of the feature matrix. Each row in the feature matrices (FIG. 6) comprises 16 radial bins, each of which has 4 angular bins. The SC images show the relative intensities of the bins, with the largest value scaled to the full byte. When a matrix is used in deep learning, it is the initially calculated values of the electronic properties that are processed.

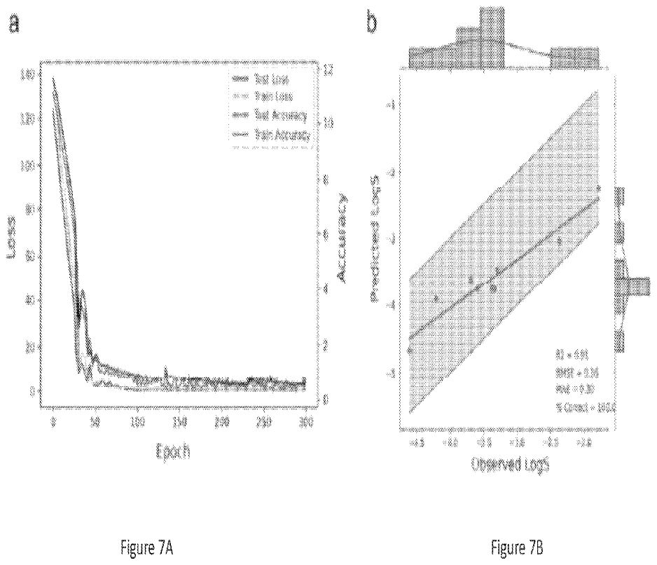

MEMS has been implemented for predicting water solubility of organic molecules by deep learning. The deep-learning effort utilized a curated dataset of about 160 molecules, which was split into 9:1 as the training and testing sets. Hirshfeld surfaces of the crystal structures of these molecules were calculated and reduced to manifold embeddings. Respective electronic properties (electron density, ESP, and Fukui functions) were evaluated of the single molecules with the conformations extracted from the individual crystals. Feature matrices were then derived by SC and used as the input for deep learning. The input of each molecule consisted of several feature matrices, including electronic density, ESP, f⁺, f¹, and f². DeepSets was adapted as the architecture of deep learning; self-attention was used as the learning mechanism in the deep neural network. PyTorch was used to implement the deep learning. The solubility prediction achieved a much-improved prediction accuracy compared with most of the reported literature studies. FIG. 7 shows one testing set's deep learning cost and prediction results where loss and accuracy of one test set of molecules during the deep learning (a) and predicted solubility (in log unit) vs. experimental values of the testing molecules (b). The mean absolute error (MAE) was 0.3 log unit, much smaller than the current prediction benchmark of 1 log unit. MEMS has also been used for predicting binding affinities of small molecules to cytochrome P450 enzymes. Computing the MEMS and SC features of more than 14,000 single molecules taken from PubChem (A1851), we may achieve similar and better prediction results of classification (active vs. inactive) compared to other reported efforts. Further analysis by regression prediction is also considered. Preliminary results support the feasibility of using MEMS of single molecules to predict intermolecular interactions.

The detailed descriptions which follow are presented in part in terms of algorithms and symbolic representations of operations on data bits within a computer memory representing genetic profiling information derived from patient sample data and populated into network models. A computer generally includes a processor for executing instructions and memory for storing instructions and data. When a general purpose computer has a series of machine encoded instructions stored in its memory, the computer operating on such encoded instructions may become a specific type of machine, namely a computer particularly configured to perform the operations embodied by the series of instructions. Some of the instructions may be adapted to produce signals that control operation of other machines and thus may operate through those control signals to transform materials far removed from the computer itself. These descriptions and representations are the means used by those skilled in the art of data processing arts to most effectively convey the substance of their work to others skilled in the art.

An algorithm is here, and generally, conceived to be a self-consistent sequence of steps leading to a desired result. These steps are those requiring physical manipulations of physical quantities. Usually, though not necessarily, these quantities take the form of electrical or magnetic pulses or signals capable of being stored, transferred, transformed, combined, compared, and otherwise manipulated. It proves convenient at times, principally for reasons of common usage, to refer to these signals as bits, values, symbols, characters, display data, terms, numbers, or the like as a reference to the physical items or manifestations in which such signals are embodied or expressed. It should be borne in mind, however, that all of these and similar terms are to be associated with the appropriate physical quantities and are merely used here as convenient labels applied to these quantities.

Some algorithms may use data structures for both inputting information and producing the desired result. Data structures greatly facilitate data management by data processing systems, and are not accessible except through sophisticated software systems. Data structures are not the information content of a memory, rather they represent specific electronic structural elements which impart or manifest a physical organization on the information stored in memory. More than mere abstraction, the data structures are specific electrical or magnetic structural elements in memory which simultaneously represent complex data accurately, often data modeling physical characteristics of related items, and provide increased efficiency in computer operation.

Further, the manipulations performed are often referred to in terms, such as comparing or adding, commonly associated with mental operations performed by a human operator. No such capability of a human operator is necessary, or desirable in most cases, in any of the operations described herein which form part of the present invention; the operations are machine operations. Useful machines for performing the operations of the present invention include general purpose digital computers or other similar devices. In all cases the distinction between the method operations in operating a computer and the method of computation itself should be recognized. The present invention relates to a method and apparatus for operating a computer in processing electrical or other (e.g., mechanical, chemical) physical signals to generate other desired physical manifestations or signals. The computer operates on software modules, which are collections of signals stored on a media that represents a series of machine instructions that enable the computer processor to perform the machine instructions that implement the algorithmic steps. Such machine instructions may be the actual computer code the processor interprets to implement the instructions, or alternatively may be a higher level coding of the instructions that is interpreted to obtain the actual computer code. The software module may also include a hardware component, wherein some aspects of the algorithm are performed by the circuitry itself rather as a result of an instruction.

The present invention also relates to an apparatus for performing these operations. This apparatus may be specifically constructed for the required purposes or it may comprise a general purpose computer as selectively activated or reconfigured by a computer program stored in the computer. The algorithms presented herein are not inherently related to any particular computer or other apparatus unless explicitly indicated as requiring particular hardware. In some cases, the computer programs may communicate or relate to other programs or equipment through signals configured to particular protocols which may or may not require specific hardware or programming to interact. In particular, various general purpose machines may be used with programs written in accordance with the teachings herein, or it may prove more convenient to construct more specialized apparatus to perform the required method steps. The required structure for a variety of these machines will appear from the description below.

The present invention may deal with “object-oriented” software, and particularly with an “object-oriented” operating system. The “object-oriented” software is organized into “objects”, each comprising a block of computer instructions describing various procedures (“methods”) to be performed in response to “messages” sent to the object or “events” which occur with the object. Such operations include, for example, the manipulation of variables, the activation of an object by an external event, and the transmission of one or more messages to other objects.

Messages are sent and received between objects having certain functions and knowledge to carry out processes. Messages are generated in response to user instructions, for example, by a user activating an icon with a “mouse” pointer generating an event. Also, messages may be generated by an object in response to the receipt of a message. When one of the objects receives a message, the object carries out an operation (a message procedure) corresponding to the message and, if necessary, returns a result of the operation. Each object has a region where internal states (instance variables) of the object itself are stored and where the other objects are not allowed to access. One feature of the object-oriented system is inheritance. For example, an object for drawing a “circle” on a display may inherit functions and knowledge from another object for drawing a “shape” on a display.

A programmer “programs” in an object-oriented programming language by writing individual blocks of code each of which creates an object by defining its methods. A collection of such objects adapted to communicate with one another by means of messages comprises an object-oriented program. Object-oriented computer programming facilitates the modeling of interactive systems in that each component of the system may be modeled with an object, the behavior of each component being simulated by the methods of its corresponding object, and the interactions between components being simulated by messages transmitted between objects.

An operator may stimulate a collection of interrelated objects comprising an object-oriented program by sending a message to one of the objects. The receipt of the message may cause the object to respond by carrying out predetermined functions which may include sending additional messages to one or more other objects. The other objects may in turn carry out additional functions in response to the messages they receive, including sending still more messages. In this manner, sequences of message and response may continue indefinitely or may come to an end when all messages have been responded to and no new messages are being sent. When modeling systems utilizing an object-oriented language, a programmer need only think in terms of how each component of a modeled system responds to a stimulus and not in terms of the sequence of operations to be performed in response to some stimulus. Such sequence of operations naturally flows out of the interactions between the objects in response to the stimulus and need not be preordained by the programmer.

Although object-oriented programming makes simulation of systems of interrelated components more intuitive, the operation of an object-oriented program is often difficult to understand because the sequence of operations carried out by an object-oriented program is usually not immediately apparent from a software listing as in the case for sequentially organized programs. Nor is it easy to determine how an object-oriented program works through observation of the readily apparent manifestations of its operation. Most of the operations carried out by a computer in response to a program are “invisible” to an observer since only a relatively few steps in a program typically produce an observable computer output.

In the following description, several terms which are used frequently have specialized meanings in the present context. The term “object” relates to a set of computer instructions and associated data which may be activated directly or indirectly by the user. The terms “windowing environment”, “running in windows”, and “object oriented operating system” are used to denote a computer user interface in which information is manipulated and displayed on a video display such as within bounded regions on a raster scanned video display. The terms “network”, “local area network”, “LAN”, “wide area network”, or “WAN” mean two or more computers which are connected in such a manner that messages may be transmitted between the computers. In such computer networks, typically one or more computers operate as a “server”, a computer with large storage devices such as hard disk drives and communication hardware to operate peripheral devices such as printers or modems. Other computers, termed “workstations”, provide a user interface so that users of computer networks may access the network resources, such as shared data files, common peripheral devices, and inter-workstation communication. Users activate computer programs or network resources to create “processes” which include both the general operation of the computer program along with specific operating characteristics determined by input variables and its environment. Similar to a process is an agent (sometimes called an intelligent agent), which is a process that gathers information or performs some other service without user intervention and on some regular schedule. Typically, an agent, using parameters typically provided by the user, searches locations either on the host machine or at some other point on a network, gathers the information relevant to the purpose of the agent, and presents it to the user on a periodic basis. A “module” refers to a portion of a computer system and/or software program that carries out one or more specific functions and may be used alone or combined with other modules of the same system or program.

The term “desktop” means a specific user interface which presents a menu or display of objects with associated settings for the user associated with the desktop. When the desktop accesses a network resource, which typically requires an application program to execute on the remote server, the desktop calls an Application Program Interface, or “API”, to allow the user to provide commands to the network resource and observe any output. The term “Browser” refers to a program which is not necessarily apparent to the user, but which is responsible for transmitting messages between the desktop and the network server and for displaying and interacting with the network user. Browsers are designed to utilize a communications protocol for transmission of text and graphic information over a world wide network of computers, namely the “World Wide Web” or simply the “Web”. Examples of Browsers compatible with one or more embodiments of the present invention include the Chrome browser program developed by Google Inc. of Mountain View, California (Chrome is a trademark of Google Inc.), the Safari browser program developed by Apple Inc. of Cupertino, California (Safari is a registered trademark of Apple Inc.), Internet Explorer program sold by Microsoft Corporation (Internet Explorer is a trademark of Microsoft Corporation), the Opera Browser program created by Opera Software ASA, or the Firefox browser program distributed by the Mozilla Foundation (Firefox is a registered trademark of the Mozilla Foundation). Although the following description details such operations in terms of a graphic user interface of a Browser, the present invention may be practiced with text based interfaces, or even with voice or visually activated interfaces, that have many of the functions of a graphic based Browser.

Browsers display information which is formatted in a Standard Generalized Markup Language (“SGML”) or a HyperText Markup Language (“HTML”), both being scripting languages which embed non-visual codes in a text document through the use of special ASCII text codes. Files in these formats may be easily transmitted across computer networks, including global information networks like the Internet, and allow the Browsers to display text, images, and play audio and video recordings. The Web utilizes these data file formats to conjunction with its communication protocol to transmit such information between servers and workstations. Browsers may also be programmed to display information provided in an extensible Markup Language (“XML”) file, with XML files being capable of use with several Document Type Definitions (“DTD”) and thus more general in nature than SGML or HTML. The XML file may be analogized to an object, as the data and the stylesheet formatting are separately contained (formatting may be thought of as methods of displaying information, thus an XML file has data and an associated method). Similarly, JavaScript Object Notation (JSON) may be used to convert between data file formats.

The terms “personal digital assistant” or “PDA”, as defined above, means any handheld, mobile device that combines computing, telephone, fax, e-mail and networking features. The terms “wireless wide area network” or “WWAN” mean a wireless network that serves as the medium for the transmission of data between a handheld device and a computer. The term “synchronization” means the exchanging of information between a first device, e.g. a handheld device, and a second device, e.g. a desktop computer, either via wires or wirelessly. Synchronization ensures that the data on both devices are identical (at least at the time of synchronization).

Data may also be synchronized between computer systems and telephony systems. Such systems are known and include keypad based data entry over a telephone line, voice recognition over a telephone line, and voice over internet protocol (“VoIP”). In this way, computer systems may recognize callers by associating particular numbers with known identities. More sophisticated call center software systems integrate computer information processing and telephony exchanges. Such systems initially were based on fixed wired telephony connections, but such systems have migrated to wireless technology.

In wireless wide area networks, communication primarily occurs through the transmission of radio signals over analog, digital cellular or personal communications service (“PCS”) networks. Signals may also be transmitted through microwaves and other electromagnetic waves. At the present time, most wireless data communication takes place across cellular systems using second generation technology such as code-division multiple access (“CDMA”), time division multiple access (“TDMA”), the Global System for Mobile Communications (“GSM”), Third Generation (wideband or “3G”), Fourth Generation (broadband or “4G”), personal digital cellular (“PDC”), or through packet-data technology over analog systems such as cellular digital packet data (CDPD”) used on the Advance Mobile Phone Service (“AMPS”).

The terms “wireless application protocol” or “WAP” mean a universal specification to facilitate the delivery and presentation of web-based data on handheld and mobile devices with small user interfaces. “Mobile Software” refers to the software operating system which allows for application programs to be implemented on a mobile device such as a mobile telephone or PDA. Examples of Mobile Software are Java and Java ME (Java and JavaME are trademarks of Sun Microsystems, Inc. of Santa Clara, California), BREW (BREW is a registered trademark of Qualcomm Incorporated of San Diego, California), Windows Mobile (Windows is a registered trademark of Microsoft Corporation of Redmond, Washington), Palm OS (Palm is a registered trademark of Palm, Inc. of Sunnyvale, California), Symbian OS (Symbian is a registered trademark of Symbian Software Limited Corporation of London, United Kingdom), ANDROID OS (ANDROID is a registered trademark of Google, Inc. of Mountain View, California), and iPhone OS (iPhone is a registered trademark of Apple, Inc. of Cupertino, California), and Windows Phone 7. “Mobile Apps” refers to software programs written for execution with Mobile Software.

“Machine Learning,” “Artificial Intelligence,” and related terms which relate to “Deep Learning” involve using data sets in a convolutional neural network/machine learning environment, wherein various quantum chemistry features of molecules are classified by using training and validation sets. Convolutional neural network architectures, for example without limitation, DenseNet, Inception V3, VGGNet, ResNet and Xception, may be configured for use with MEMS data structures and models. Typically, detection and identification systems are implemented in 3 phases consisting of data collection, development of the neural network, and assessment/re-assessment of the network. Development of the neural network involves selecting the optimal network design and subsequent training the “final” model, which is then assessed using the unseen test and validation sets. Training of the neural network is an automatic process, which is continued until validation loss plateaus. Training may be augmented with additional data sets and model manipulations. Coding may be implemented, for example without limitation, using the Python programming language with the Tensorflow and Keras machine learning frameworks. Training may be performed on GPUs such as made by nVIDIA Inc. of Santa Clara, California.

FIG. 6 illustrates the featurization scheme of Shape Context on MEMS. The process is aimed to reduce the dimensions of MEMS from the number of pixels of the 2-D embedding to the number of bins per keypoint on the embedding. The scheme produced advantages in the preliminary studies, corroborating that electronic density and properties on a molecular surface do not typically bear complex patterns but center on a few surface points near the closest atoms of the molecule. Additionally, transitions between major electronic spots on a molecular surface are smooth. The molecular surfaces in FIG. 3 exemplify the global electronic patterns.

Accordingly, analytical functions are presented that express MEMS and utilize the parameters of the functions to represent MEMS for deep learning. The radial basis function (RBF) interpolation method used in generating MEMS figures of electronic properties allows for recovering major electronic patterns on a 3-D molecular surface, with Gaussian Process as Gaussian kernels being used in both approaches. While RBF interpolation relies on finding the mixing weights of several Gaussian kernels (and often supplemented by polynomials) on scattered data points, GP is much more flexible and powerful to sample an infinite number of points as random functions that share a joint, underlying probability (Gaussian) distribution. Moreover, because the electronic properties of a molecule are collectively defined by the atoms and their chemical bonds, GP is highly appealing to capture the chemical intuition by describing the distribution of the electronic properties. To curb COD, GPLVM is further used to collectively reduce the dimensionality of a set of MEMS in a low-dimensional latent space.

GP of MEMS: Being a functional, GP regulates the mean and covariance functions as the normal distribution. Interpolation of the embedding points of MEMS may be treated as GP regression through the Bayes' Theorem. Briefly, given N training data points (X, Y) (i.e., the likelihood)—where X is the position vector, and Y is the mean value vector—the values at N. testing position X_{*} (the posterior) are estimated by Y_{*}=K_{*}^{T}K⁻¹Y, where K_{*} is the N×N_{*} covariance matrix among the testing and training data points, and K is the N×N covariance matrix among the training data points. As the value at a data point is treated as a Gaussian, the variance at each testing point is estimated by the covariance matrices. Moreover, each element in the covariance matrices is a kernel function, and the Gaussian kernel (also known as squared exponential kernel or RBF kernel) is commonly used:

$$κ ( {x}_{i} , {x}_{j} ) = {σ}^{2} exp ( - \frac{1}{2 {l}^{2}} {( {x}_{i} - {x}_{j} )}^{T} ( {x}_{i} - {x}_{j} ) ) \qquad ( Eq . 2 )$$

where σ and l are predetermined hyperparameters controlling the vertical variance and smoothness of the GP at Y_{*}. Each kernel is determined by the distance between two data points. The mean at a testing position is a weighted regression by means of the training data; the kernel functions determine the weights (and thus the transition smoothness among the data points). Note that in MEMS, xᵢ is a two-dimensional position vector, but GP can handle multi-dimensional data.

Thus, we can interpolate the electronic properties of manifold embedding points (e.g., FIG. 4) with GP and produce full MEMS figures (e.g., FIG. 5). In this case, the embedding points of surface vertices are the training data (X, Y) and interpolating positions are the testing X_{*}. Nevertheless, there are typically more than 6000 surface points used in the manifold embedding process, making the GP calculation computationally demanding (mainly due to the inversion operation of K, which becomes intractable for larger N). Moreover, using a significantly large number of GP parameters (of the training data) to express the generally simple electronic patterns on a molecular surface is unnecessary. It deviates from our purpose of using GP parameters to represent MEMS. For this, we will use sparse GP (SGP) instead.

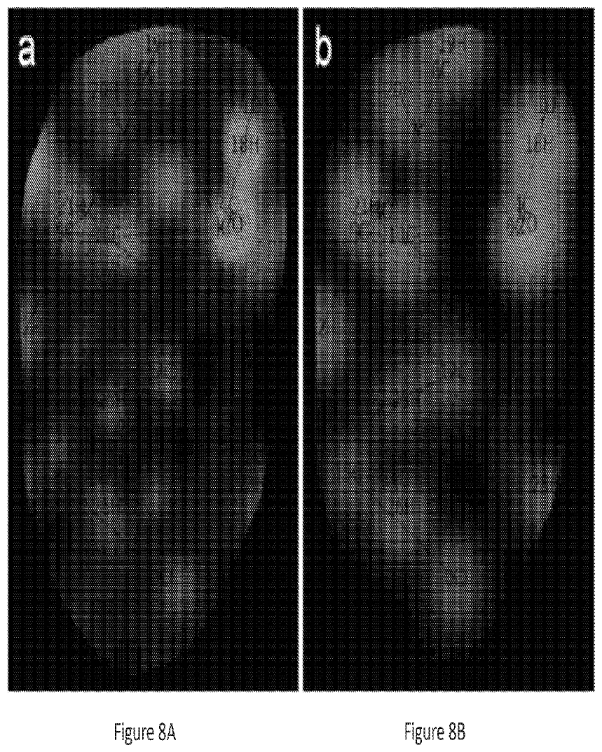

In SGP, rather than using the full training data points, a limited number of “inducing” points are selected, Xₘ. Then, the optimal Yₘ and Kₘₘ (covariance matrix of the inducing points) can be found by minimizing the KL divergence between the approximate and true Y of the training data. Mean values of the testing data are given as follows: Y*=K^{T}*ₘ K⁻¹ₘₘ Yₘ, similarly to the regular GP. In our case, we will use the key points that we did with SC (FIG. 6), i.e., for each atom, we select the closest vertex on the molecular surface as an inducing point. The number of Xₘ is then equal to the number of atoms. We will conduct KL divergence optimization to derive Yₘ and Kₘₘ with the embedding points as Y. FIG. 8, showing MEMS generated by RBF interpolation of all embedding points (a) and approximated MEMS by SGP (b), illustrates a preliminary attempt at using SGP to interpolate a MEMS. The result supports the feasibility and suggests several areas to explore.

In FIG. 8b, the isotropic Gaussian kernel was used with the same length parameter, l, to calculate the distance along the x and y axes between two points (Eq. 2). The same parameter was used for all inducing points. In setting up this molecular model, the parameters in different directions and identify optimized l (and σ) by variational inference. The hyperparameters may be eventually linked to the underlying atomic type and bonding for each including point.

There are “spots” between inducing points (including the spots near the boundary) that were not picked up. These spots are likely due to unique chemical bonding, such as aromatic conjugation. To accommodate, different types of kernel functions and their combinations may be used.

As discussed above, MEMS of a closed molecular surface results in false negatives and positives. A substantial spot on a surface may lead to more than one spot on the MEMS. For dealing with a closed surface, it is worth exploring at least two points for each atom on the 2-D embedding. Rotational invariance is implicitly ensured because of the GP kernels are distance-based.

Thus, the parameters used to express MEMS are used as input for deep learning. At least three parameters representing each inducing point (two position numbers and mean value) of one electronic property. As discussed above, a few more parameters may be included to consider the anisotropic and inbetween patterns. Compared to the 64 bins used in FIG. 7, using the GP parameters considerably reduces the dimensionality of MEMS. Hyperparameters, including l and o that are established according to the atomic and bonding information, could further reduce dimensionality (by using the atomic number to replace kernel parameters). Interpreting MEMS with SGP may potentially establish the connection between MEMS and the underlying chemistry (and molecule), facilitating the reserve mapping from MEMS to molecular structure.

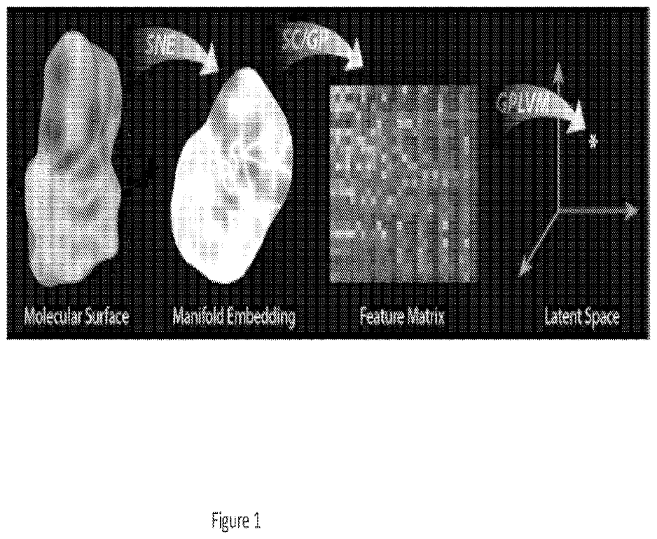

GPLVM of MEMS FEATURE: Even with the featurization of SC and GP, the dimensionality of MEMS remains significant. As indicated by FIG. 1, we aim to reduce MEMS lastly—whether it is signified as an image (e.g., FIG. 5), SC matrix (e.g., FIG. 6), or GP parameter matrix—into a low-dimensional latent space. The dimensionality reduction may be implemented by GP Latent Variable Model. The method is not targeted to one MEMS, but a collection of MEMS calculated from a dataset of molecules.

GPLVM was developed out of the probabilistic principal component analysis (PCA). Each data dimension is treated as a GP of the (unknown) latent variables, and all the independent GPs are collected and optimized to derive the latent variables (and hyperparameters). Let Y∈R^{nxp} be n MEMS feature matrices with p dimensions and X∈R^{n×d} n latent variables with d<<p that are mapped to Y by GPs. Specifically, the latent variables are derived by maximizing the marginal likelihood defined below:

$$P ( Y \vert X , Φ ) = {\prod }_{j = 1}^{p} \frac{1}{{( 2 π )}^{\frac{n}{2}} {\vert K \vert}^{\frac{1}{2}}} exp ( - \frac{1}{2} {y}_{j}^{T} {K}^{- 1} {y}_{j} ) \qquad ( Eq . 3 )$$

where yⱼ is the j^{th} dimension of the data, K is the covariance matrix by X, and Φ are hyperparameters used by kernel functions.

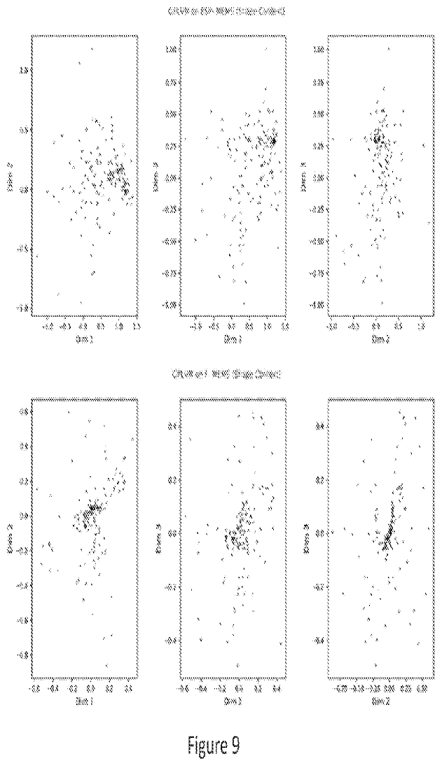

FIG. 9 illustrates the latent variables with three dimensions (d=3) of 162 MEMS feature matrices of negative ESP and electrophilic Fukui function (f-), respectively, from our solubility prediction study (FIG. 7). In FIG. 9, MEMS (shape context) of 162 molecules are reduced to 3-D latent spaces by GPLVM, wherein the top row is based on ESP; the bottom on f-(three 2-D projections of the latent spaces are shown respectively). Each SC matrix has a dimension of 2304 (36 atoms times 64 bins; matrices of molecules with fewer atoms are padded with zeros) and is reduced by GPLVM to a single point in the latent space. It is evident that even with d=3, the high-dimensional MEMS are individually differentiated and uniquely signified in one chemical space. In this case, each molecule may be represented by only six dimensions (or 12 when positive ESP and f+ are included). The feat is remarkable in chemical learning, as this allows for systemically projecting molecules in a low-dimensional space defined by quantum chemical dimensions. Given that SGP can be used in GPLVM, it is practically feasible to reduce millions of molecules (as MEMS) to a discriminative latent space. Moreover, because GPLVM is a generative model, any point in the latent space may be derived back to MEMS, opening a door for de novo design.

In one embodiment, a variational inference approach is used to optimize kernel parameters (Φ) and latent dimension (d),67 before obtaining the latent variables (X). In addition, a Bayesian scheme to predict the latent variable for a new data (MEMS matrix) after the GPLVM latent space is established. This procedure is improves property prediction when a (deep learning) model is trained with available data, and maximizes the extent to which the chemistry is captured by manifold embedding.

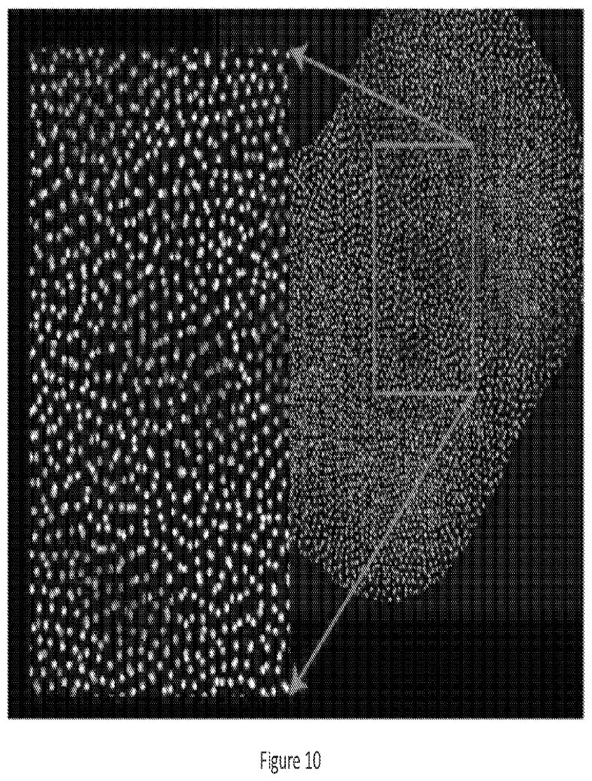

In FIG. 10 a molecular surface is enclosed, showing surface points on MEMS with the electronic property being f². When calculating the geodesic distance between two surface points (or vertices of surface mesh), the shortest one is used in defining the neighborhood of a point (Eq. 1). However, the KL optimization process generates some false positives (distant neighbors in 3-D put in the same neighborhood on MEMS) and false negatives (near neighbors in 3-D separated on MEMS) on a final manifold embedding. This is illustrated in FIG. 10, where several blue, red, and white points are projected near each other in the highlighted area. On the original molecular surface, these points belong to different regions of f² values. The ambiguity raises several questions in general for deep learning. First, how much information is lost by the dimensionality reduction process? Second, how to eliminate the generated false information and still be able to utilize the remaining data for prediction? Last, how to minimize the generation of false positives and negatives?

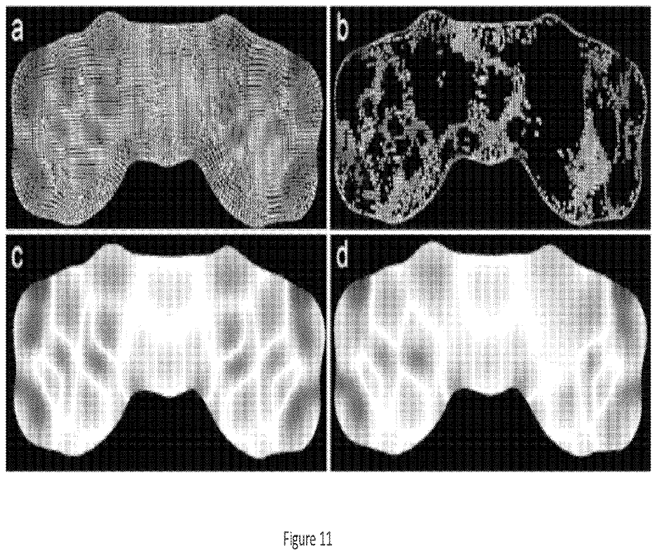

To address the first question, a basic scheme is used to estimate the percent of surface points in a “wrong” neighborhood. By defining the neighborhood size twice larger than the shortest inter-point distance on the surface or MEME, a point may be assigned as “outsider” if none of its MEMS neighbors comes from its neighborhood on the 3-D surface. Typically, the percent of outsiders is in the range of 20-40%, depending on the geometry of the molecular surface (and initial positions for KL optimization). For the second question, by using RBF to interpolate the points on MEMS and generate final MEMS images, the major or dominant electronic patterns and their spatial relationships from the original molecular surface are preserved on MEMS. FIG. 11 highlights one extreme case where 3247 points were kept out of 9011 embedding points but still led to the recovery of major electronic patterns by RBF interpolation. Ostensibly, these results suggest that the Gaussian kernels used by RBF smooth out data “noises” of false positives and negatives. Deeply, the recovery of dominant electronic features on MEMS implies a rationale in the inventive process: the underlying dimensionality of electronic attributes on MEMS is much smaller than that of MEMS itself (as a matrix or image format). The rationale is not intuitively surprising, as electronic features on a molecular surface smoothly spread over domains that are commensurate with the size of an atom.

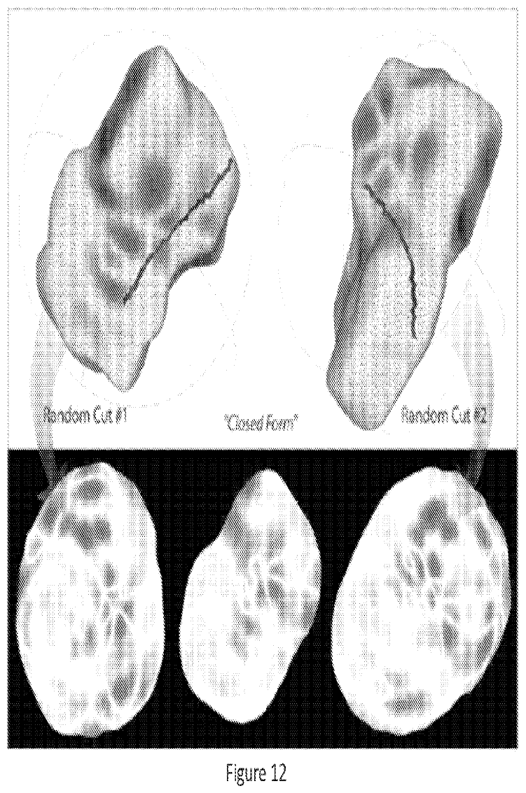

The ambiguity due to false positives and negatives may still result in uncertainties in deep learning of chemical data. This may be exemplified when the Earth globe is projected onto a World map—the “Far East” on one map becomes the “Central Kingdom” on another. To minimize the false information in MEMS, one method involves cutting a molecular surface by removing the connectivity between surface vertices along a randomly chosen surface line. The cutting forces the vertices to be the boundary points of MEMS while keeping other surface points in the right neighborhood. For example, FIG. 12 shows MEMS of the same surface (f²) randomly cut, where both open-cut MEMS have no point in a wrong neighborhood, showing dimensionality reduction of the same manifold being cut with the middle embedding being of the closed manifold. In comparison, the closed MEMS has about 40% false points. The results are exciting and significant, supporting that the manifold embedding truthfully preserves the neighborhood information (of the cut surface). The closed MEMS still retains a good portion of major electronic distributions. Nonetheless, the cutting was done randomly and may not lead to the maximal preservation of the quantum chemical information on a surface. Another method that may be used is Geodesic Principal Analysis (PGA) to choose a surface direction for cutting. In brief, PGA firstly identifies an “intrinsic mean” on the surface of interest that has the minimal total geodesic distances to all surface points. Then, the surface points are projected to the tangential plane at the mean, and PCA is done on the projected points. Lastly, the identified principal vectors on the tangential space are projected back to the surface (manifold) as the principal geodesics. In our case, we will have two orthonormal principal vectors yielded from the Euclidean space (on the tangential plane) and, subsequently, two principal geodesics on a molecular surface; by surface cutting along the principal geodesics and generate respective MEMS. The first principal geodesics may be a better choice of cutting as surface points collectively have larger variances along the direction. A molecule may be represented using both open-cut MEMS in deep learning.

Furthermore open-cut MEMS may be used for solubility prediction of organic molecules. To enhance the reliability of solubility prediction, cutting is done not randomly but between two surface points intercepted by principal axes of mesh points. As revealed in FIG. 7, the prediction accuracy was well under 1 log unit, a state-of-the-art standard currently observed by the community. Further improvement by using MEMS of cut surfaces may be achieved. In addition, the same deep learning framework with MEMS may be used to predict the membrane permeability of molecules. In addition to solubility, permeability is another key quantity determining the bioavailability of a drug molecule. In initial work, more than 3,000 Caco-2 permeability measurements from ChEMBL have been collected. Then curating the dataset, calculating electronic properties on these molecules' surface, and obtaining MEMS of both cut and closed surfaces may be used, featurized by SC and GP, as the input for deep learning. Moreover, low-dimensional latent representations of these MEMS may be derived by GPLVM and further exploited for deep learning as well. Lastly, MEMS of major conformers of each molecule (see below) may be tested for permeability prediction.

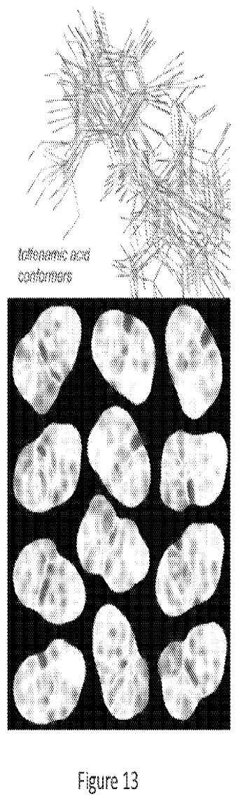

In preliminary studies, only either the most stable conformer (optimized by Gaussian 09 in vacuum or implicit solvent) or the conformer taken directly from the crystal structure of a molecule have been considered. This approach works well for molecular properties where such conformers are most relevant (e.g., solubility being a crystal property). However, the conformational flexibility of a molecule may also be considered for predicting molecular interactions such as protein-ligand binding, where the conformational energy is a co-factor. Because MEMS is already in 2D and readily featurized to a mathematic matrix (by SC or GP), integrating MEMS of the major conformers of a molecule is straightforward and improves the predictive power of a deep learning model significantly. FIG. 13 shows MEMS of the major MEMS of the molecular surfaces (f²) of major conformers of tolfenamic acid, generated by ConfGen (Schrödinger Co.). Thus, stacking the conformers' feature matrices as the molecular representation is made in several embodiments of the invention. Conformational energies may be further used to weigh or normalize the respective matrices. This enables much-improved prediction results for studying molecular interactions and pertinent properties.

In one embodiment, prediction utilizes the conformer's MEMS for predicting binding activities to cytochrome P450 enzymes (CYP450). We have conducted preliminary studies with a publicly available dataset on PubChem (A1851), which reports binding assay results of molecules with six isoforms (1A2, 2B6, 2C9, 2C19, 2D6, and 3A4). The reported activity score is in the range of [0, 100], with the cutoff of 40 indicating a compound being active or inactive against a CYP450 enzyme. By selecting drug-like molecules, we ended up with 14,567 unique molecules from the database and trained our deep-learning models. The most stable conformer of each molecule was identified and calculated by Gaussian 09; electronic properties on the surface were mapped to its MEMS and featurized by SC. Our classification prediction (activity vs. inactive) showed comparable and better results compared with the literature-reported data. For example, the F-score was 0.87, and the accuracy was 0.79 for 1A2. On the other hand, our regression prediction resulted in mediocre MAE (mean absolute error) between 10 and 20 of activity scores. Much-improved prediction, especially the regression to have the MAE less than 10, may be achieved by considering the conformational flexibility of the molecules. By generating and selecting a predetermined number of major conformers of a molecule and calculating their MEMS, predictive quality improves. In addition, predictions of human microsomal clearance may be accomplished by using a curated dataset (>5,300 data points).

Chemical information determining both the strength and specificity of molecular interactions are carried by the molecular representation for predicting ligand binding with a protein. In the classification prediction of CYP450 binding, the MEMS used (of closed surfaces and without conformers) performed satisfactory, likely due to the binding strength being decided by the dominant electronic attributes that are retained in MEMS. However, for the regression prediction, complete information is helpful, especially about the spatial distribution of the electronic properties, along with the conformational dimension.

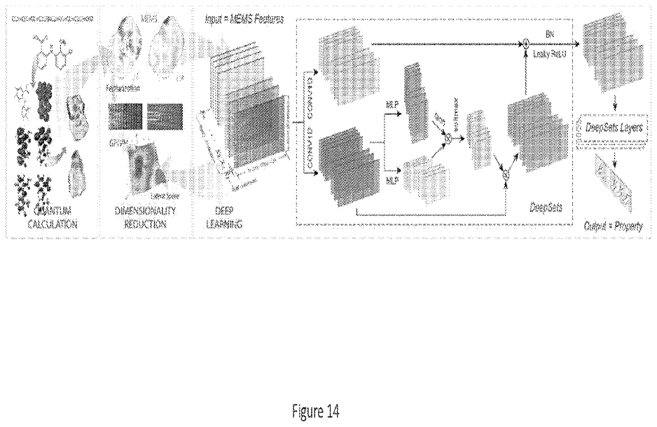

FIG. 14 shows one embodiment of a prediction framework of chemical properties (including affinity with protein target) integrating three parts: quantum calculation, dimensionality reduction, and deep learning. The process begins with obtaining a given molecule's conformer(s), followed by electronic calculation and generation of the molecular surface(s) of local electronic attributes. MEMS is then derived and featurized by SC or GP. Feature matrices of training molecules are projected to a low-dimensional space by GPLVM. MEMS features or latent variables of the training molecules are input to the deep learning module, in which DeepSets is adopted as the architecture. The input consists of a batch of molecules, each of which has several MEMS feature matrices of electronic properties. Multiple DeepSets layers are utilized, leading to the output of the network. With the training data of the property to be predicted, backpropagation is conducted by stochastic gradient descent leading to the optimization of network parameters.

Virtual screening applications of small molecules may be developed using the MEMS data structure and model. The general workflow of deep learning for the applications is highlighted in FIG. 14. These applications are summarized in Table 1. While the Universal Approximation Theorem suggests that artificial neural networks may approximate or learn any function, it does not reveal how a neural network is implemented. The performance of a deep learning model is governed by the network architecture, definition of the loss function, and quality of the training data. Equally important is whether the input contains sufficient information but with low-dimensional features to capture the underlying function. In a typical chemical learning of molecular property or function, the input is of a batch of molecules, in which each variable consists of feature vectors of the molecule's constituent atoms. Such a variable is a set where the ordering of atomic elements should be irrelevant for its functions to be learned. In this exemplary embodiment, DeepSets has been adopted as the deep-learning architecture. It is permutation invariant and potentially may learn any set function by a sum-decomposition scheme. In brief, the input is first transformed to a latent space—in this exemplary case, it is done by CONV1D along the feature dimension—and before transforming back to the output, the latent variables (or tensors) are processed by sum (or mean or max) operations to remove (or retain) the ordering information of the input set. Specifically, we have utilized the self-attention approach as the sum-decomposition that is first proposed in Set2Graph. The attention architecture (FIG. 14) may be summed as:

$$\sum ( X ) = softmax ( \frac{tanh ( {f}_{1} ( X ) ) \cdot {{f}_{2} ( X )}^{T}}{{d}^{1 / 2}} ) \cdot X \qquad ( Eq . 4 )$$

where X is the input set, d is the feature dimension of X divided by a predetermined number (typically 10), and f₁ and f₂ are the query and key functions of self-attention, which are implemented by MLP or multilayer perceptron in our models. Notably, the self-attention mechanism can be easily shown to be permutation invariant and is widely used to capture the intra-correlations of the input features, making DeepSets suitable choice for our tasks. Additionally, regularization of each DeepSets may be done by batch normalization (BN) and Leaky ReLU; weight decay is also considered in the PyTorch optimizer to further mitigate model overfitting.

Table 1 of the Appendix lists the deep-learning applications made possible by embodiments of the present invention. The selection of the applications is not meant to be comprehensive but aimed to test the MEMS concept thoroughly. The properties include a single physical process (e.g., dissolution), binding to a single protein target (e.g., CYP450), permeating through the cell membrane, or undergoing much more convoluted processes such as cell-based target-binding. The DILI database is curated from the drug labeling and clinical observations, presenting a challenging case for deep learning due to data noises and multiple in vivo events leading to DILI. A specific implementation involves carefully going through each database and focusing on drug-like molecules (neutral and with molecular weight <600 Da).

In one embodiment, completing the deep-learning exercise may take up to one year of computational time with conventional research computing resources. On a typical computer node with 20 cores, it takes less than 10 minutes to do the quantum calculation and dimensionality reduction (i.e., the first two modules shown in FIG. 14) of a single molecule. Both Gaussian 09 and our C++ programs of manifold embedding are fully parallelized; the process of launching and processing the input and output of various programs is fully streamlined by shell scripts. With 24 nodes, one may create the MEMS features of more than 10,000 molecules per week. Implementing deep learning by PyTorch with CUDA; for example, in our classification prediction of CYP450 binding, it typically takes less than 4 hours on an NVIDIA A100 GPU to process >12,000 molecules with about 11 million neural network training parameters of 9 DeepSets layers. Thus, the deep-learning projects listed in Table 1 may take two years by typical research group.

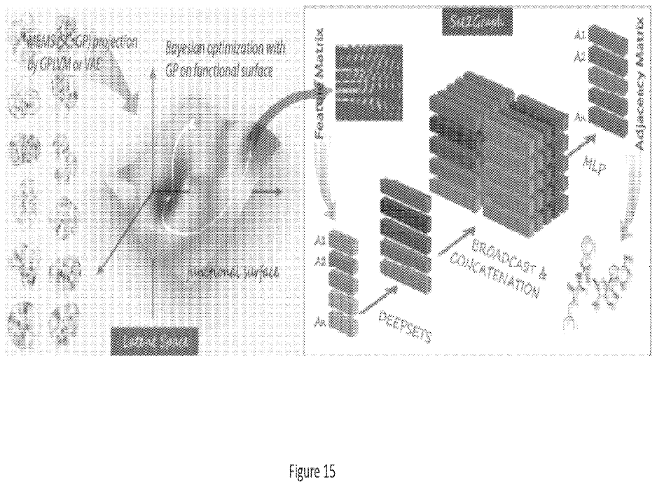

FIG. 15 illustrates the process and system of de novo development. Two components of the de novo design framework are shown: MEMS as feature matrices are firstly projected to a latent space by GPLVM or VAE; Bayesian optimization of the latent variables regarding a particular property is conducted to identify the best MEMS features; and a deep learning model based on Set2Graph is established to predict the adjacency matrix of a molecule from a given MEMS feature matrix.

GPLVM may be used to project MEMS feature matrices of molecules, which are generated by SC or GP, to a latent space. MEMS as an image (e.g., in FIG. 12) may also be projected directly, but its true dimensionality is much smaller than that of the image itself. Much fewer GP parameters of a MEMS may be capable of fully representing the electronic properties on a molecular surface.

In addition, VAE may be used to project MEMS into a latent space. Unlike GPLVM, which is a nonparametric method, VAE relies on neutral networks (and associated trainable parameters) to reduce dimensionality. VAE utilizes a multivariate Gaussian function to regularize the latent data structure to approximate the latent space (via Variation Interference). The encoder may be regarded as projecting the input data onto a latent Gaussian manifold, whose mean and covariance functions are trained by adjusting the neural network parameters via the decoder. In practice, each dimension of the Gaussian distribution is considered independent (i.e., the off-diagonal elements of the covariance matrix are treated as zero).

Both methods offer their own advantages and disadvantages. GPLVM provides more expression because of various choices of kernel functions and consideration of the full covariance matrix. It is, however, computationally expensive, especially for processing a large amount of data (mainly due to the inversion of the covariance matrix). Sparse GP may help but relies on the posterior approximation by variational inference. Importantly, GPLVM works directly on the (unknown) latent variables and has little or no capability to extract features from “raw” data. Conversely, VAE utilizes neutral networks and various architectures (e.g., CNN) to extract and project features to a latent space. On the other hand, with no correlation typically considered between latent variables, VAE may lead to ambiguities in recovering an input; it also suffers from the latent variables not being able to encode the input information (so-called Posterior Collapse75). Comparing using VAE to encode MEMS directly as the image format, the recovered images were fuzzy—a typical observation in deep learning, especially when the number of training data is small or moderate. Nonetheless, given the prowess of neural networks in extracting features from MEMS, VAE is a worthy alternative to construct the latent space of molecules.

Projecting MEMS of molecules into a low-dimensional latent space may potentially overcome COD. Because the smooth Gaussian functions are used to approximate the posterior, using GPLVM or VAE to conduct the dimensionality reduction can further make it effective to sample the latent space. As illustrated in FIG. 15, a functional surface may be explored in the latent space between the latent variables (X) and the molecular property of interest (Y) that is “tagged” to each data point (i.e., molecule). The (unknown) functional surface may be approximated by a GP (as surrogation function), and finding an optimal value by BO follows the Bayes' theorem. Briefly, BO is an iterative optimization method, and at the initial step, the GP posterior is established by the available latent variables projected from MEMS feature matrices. Then reiteratively, at each step, GP regression is conducted over the whole sample space (within bounds), and the best sampling point is identified and further used to update the current posterior. Finding the best sampling point is often done by Expected Improvement (as acquisition function), which compares the predicted means of all sampling points to the current best mean (of the molecular property of interest) and pick the best new point. Because GP regression is done analytically, finding the maximal point may be achieved by optimizing the acquisition function, augmented as probability distribution functions, to enable an “exploration” space around each sampling point. The reiteration stops after the best value is converged. Therefore, it is likely that BO may lead to the finding of the global maximum.

With a given MEMS matrix, the deep learning model shown in FIG. 15 is aimed to generate a weighted adjacency matrix of the atoms in a molecule. The adjacency matrix represents the graph of chemical bonds with the weights signifying the types of the bonds. Additionally the weight of a bond to denote the two atoms that form the bond may be used. For example, 11 may indicate a single bond between C—H and 12 is O—H single bond. Similar definitions may be well developed in a popular force field. Alternatively, two concurrent deep learning models may be trained to respectively output a regular adjacency matrix and a vector of the elements of the molecule. The deep learning architecture will be implemented by Set2Graph. In one embodiment involved in predicting the packing motifs in a supramolecular structure by training a Set2Graph model with a few thousand known crystal structures, f-scores greater than 0.6 were obtained, which may be further improved by using opencut MEMS and more training data. In some embodiments, however, close matching between the molecule that generates its MEMS and the predicted molecule(s) by the MEMS may not occur. A different molecule may yield similar surface electronic properties as those by another molecule. Additionally, because the molecular representation as adjacency matrix is zero-inflated, the stochastic gradient descent used to collectively optimize the training parameters of neural network layers usually fails to lead to high accuracy in predicting the adjacency matrix. Using Graph Embedding (e.g., via SkipGram) and Focal Loss has resulted in obtaining moderate improvements. Fortunately, experimental data is not required as a computed MEMS may be used to train the model (by comparing it with the molecular structure that produces the MEMS). Still, such “imperfection” may be highly beneficial for molecular design, as the model may allow some degree of hallucination. A final step of valence check may be included to validate a generated structure.

While MEMS may be transformative in chemical learning, surface embedding is just one of many ways to utilize a molecule's quantum chemical constitution to dimensionally reduce the representation of the molecules. Alternative embeddings such as an iso-surface of electron density, the Fiedler vector, projecting surface manifold on the Fiedler vector (1D), or GP directly applied to electronic quantities on a molecular surface may be used in such alternative embodiments. To complement deep learning of ligands, MEMS of the binding pocket of a protein target may be used to screen and find appropriate molecules. Further alternatives involve establishing connections by deep learning between MEMS and the Gaussian atomic orbitals of the underlying molecule. Also, there is the opposite side of COD-Blessing of Dimensionality—where “generic high-dimensional datasets exhibit fairly simple geometric properties,” when we apply MEMS in chemical learning, wherein after an initial processing of MEMS for discovery, prediction and de novo development, further dimensionality may be added back into a particular data set for optimization.

Further embodiments involve MEMS kernels which are more expressive than shape-context matrices in recovering the chemical information of the “underlying” molecule, particularly relevant for solubility prediction. Moreover, by directly kernelize the electronic attributes on a molecular surface without going through the embedding process one may avoids information loss due to the embedding. These exemplary approaches may be referred to as Manifold Kernelization of Molecular Surface (MKMS). Manifold kernels in many situations improve the accurate and salient knowledge in retaining the electronic information of a molecule, especially in generative deep learning for de novo design of molecules. While other types of molecular surfaces are processed similarly, Hirshfeld surfaces are illustrated in this disclosure. A Hirshfeld surface generated by methods found in the Tonto reference (Jayatilaka, D.; Grimwood, D. J., Tonto: A Fortran based object-oriented system for quantum chemistry and crystallography. In Computational Science-ICCS 2003, Pt Iv, Proceedings, Sloot, P. M. A.; Abramson, D.; Bogdanov, A. V.; Dongarra, J. J.; Zomaya, A. Y.; Gorbachev, Y. E., Eds. 2003; Vol. 2660, pp 142-151) in combination with vertices being further optimized by isotropic meshing using the methods of the Cignoni reference (Cignoni, P.; Callieri, M.; Corsini, M.; Dellepiane, M.; Ganovelli, F.; Ranzuglia, G. In MeshLab: an Open-Source Mesh Processing Tool, Sixth Eurographics Italian Chapter Conference, 2008; pp 129-136) are used in one embodiment of the invention. With mesh vertices being input, in this exemplary embodiment, to a C++ program to produce 2-D points of MEMS. To generate an embedding a method of Neighbor Retrieval Visualizer was used (for example, that disclosed in Venna, J.; Peltonen, J.; Nybo, K.; Aidos, H.; Kaski, S., Information retrieval perspective to nonlinear dimensionality reduction for data visualization. Journal of Machine Learning Research 2010, 11, 451-490). This specific process optimizes the distances among embedding points to preserve the local neighborhood of surface vertices. Specifically, it is evaluated as the probability of vertex j in the neighborhood of vertex i:

$${p}_{j \vert i} = \frac{exp ( - {g}_{ij}^{2} / {σ}_{i}^{2} )}{{\sum }_{k \neq i} exp ( - {g}_{ik}^{2} / {σ}_{i}^{2} )}$$

where gᵢⱼ is the geodesic distance and σᵢ is a parameter to determine the neighborhood coverage for i. A similar probability is defined by the Euclidean distance between the points i and j on the lower-dimensional embedding:

$${q}_{j \vert i} = \frac{exp ( - {d}_{ij}^{2} / {σ}_{i}^{2} )}{{\sum }_{k \neq i} exp ( - {d}_{ik}^{2} / {σ}_{i}^{2} )}$$

The cost functions consists of two weighted Kullback-Leibler (KL) divergences between the two probability distributions in order to balance false positive and negatives:

$$λ {\sum }_{i = 1}^{N} {\sum }_{{j = 1}_{j \neq i}}^{N} {p}_{j \vert i} log \frac{{p}_{j \vert i}}{{q}_{j \vert i}} + ( 1 - λ ) {\sum }_{i = 1}^{N} {\sum }_{{j = 1}_{j \neq i}}^{N} {q}_{j \vert i} log \frac{{q}_{j \vert i}}{{p}_{j \vert i}}$$

The parameter, λ, is to weight the two KL divergences; the inventors of the present invention found a value of 0.95 works well in embodiments of the invention. In addition, Ot is dynamically adjusted based on the input data (i.e., surface vertices) and the data density around each point, and compared to a “perplexity” hyperparameter, which was identified to be 30 in a particular embodiment.

Electronic properties on the molecule surface are then pointwisely transformed to the MEMS. The properties of single molecules, including electrostatic potential (ESP), nucleophilic Fukui function (F⁺), electrophilic Fukui function (F⁻), and dual descriptor of Fukui function (F²), are calculated by the known Gaussian 09 method (Gaussian, Inc., Wallingford CT). For several exemplary embodiments disclosed herein, including those molecules in the solubility prediction, their conformations were respectively extracted from their crystal structures and partially optimized only for the hydrogen atoms.

To featurize MEMS for deep learning, including Shape-context Featurization of MEMS we developed a numerical method enhancing a method disclosed in the Belongie reference (Belongie, S.; Mori, G.; Malik, J., Matching with shape contexts. Statistics and Analysis of Shapes 2006, 81-105). Show in FIG. 6, a feature matrix consists of rows of key points, which are the closest surface vertices to the respective atoms of the molecule in 3-D (denoted by atom indices on the figure). The intensities surrounding a key point on a MEMS image are spatially separated in predetermined bins along the radial direction. Each radial bin may be further divided into angular bins, where the angular direction is calculated against the geometric center to allow the rotational invariance of the feature matrix. When used in deep learning, it is the original values of the respective electronic properties that are processed. In one exemplary embodiment, each row in the feature matrices comprises 16 radial bins, each of which has 4 angular bins; on the other hand, there are 32 radial bins in FIG. 6.

In another exemplary embodiment, a deep-learning effort utilized selected 123 molecules from a curated dataset from the first and second solubility challenge tests similar to predictive models in two Llinas references (Llinas, A.; Glen, R. C.; Goodman, J. M., Solubility challenge: Can you predict solubilities of 32 molecules using a database of 100 reliable measurements? Journal of Chemical Information and Modeling 2008, 48 (7), 1289-1303; and Llinas, A.; Avdeef, A., Solubility Challenge Revisited after Ten Years, with Multilab Shake-Flask Data, Using Tight (SD similar to 0.17 log) and Loose (SD similar to 0.62 log) Test Sets. Journal of Chemical Information and Modeling 2019, 59 (6), 3036-3040), being randomly split into 9:1 as the training and testing sets. Selection of the molecules was limited to those with one molecule in the asymmetric unit (i.e., Z′=1). This exemplary embodiment involves Hirshfeld surfaces of the crystal structures of these molecules being calculated, and further dimensionality-reduced to manifold embeddings. Respective electronic properties (electron density, ESP, and Fukui functions) are then evaluated of the single molecules with the conformations extracted from respective crystals. Feature matrices are then derived by the shape context approach and used as the input for deep learning. The input of each molecule consisted of several feature matrices. DeepSets was adapted as the architecture of deep learning as proposed by the Zaheer reference (Zaheer, M.; Kottur, S.; Ravanbhakhsh, S.; Poczos, B.; Salakhutdinov, R.; Smola, A. J. In Deep Sets, 31st Annual Conference on Neural Information Processing Systems (NIPS), Long Beach, CA, December 4-09; Long Beach, CA, 2017); self-attention then used as the sum-decomposition as demonstrated in Set2Graph environment as disclosed in the Vaswani reference (Vaswani, A.; Shazeer, N.; Parmar, N.; Uszkoreit, J.; Jones, L.; Gomez, A. N.; Kaiser, L.; Polosukhin, I. In Attention Is All You Need, 31st Annual Conference on Neural Information Processing Systems (NIPS), Long Beach, CA, December 4-09; Long Beach, CA, 2017) then modified to consider intermolecular direct contacts.

In an alternative embodiment, the attention architecture may be described as follows:

$$\sum ( X ) = softmax ( \frac{tanh ( {f}_{1} ( X ) ) \cdot {{f}_{2} ( X )}^{T}}{{d}^{1 / 2}} ) \cdot X$$

where X is the input set of MEMS features, dis the feature dimension of X divided by a predetermined number (typically 10), and f₁ and f₂ are the query and key functions of self-attention, which are implemented by MLP or multilayer perceptron. A consists of adjacency matrices of the molecules, which donate the close contacts between the atoms of adjacency molecules in crystal. Notably, the self-attention mechanism is permutation invariant and is widely used to capture the intra-correlations of the input features. Additionally, regularization of each DeepSets is done by batch normalization (BN) and Leaky ReLU; weight decay and dropout (typically set at 50%) are also considered in the PyTorch optimizer to further mitigate model overfitting. When five 4×16 shape-context matrices were used for each molecule (including electron density, positive ESP, negative ESP, F⁺, and F⁻), there were 320 dimensions of each key point (or atom). In one exemplary embodiment of the invention, 12 DeepSets layers of (512, 512, 256, 256, 64, 64, 32, 32, 16, 16, 4, 4) feature dimensions are used, with Learning rate being set at 0.0001, L1 loss chosen as the cost function, all optimized by the Adam algorithm in PyTorch.

Embodiments of the invention provide modeling for molecules that more accurately predict chemical behavior. When molecules (in a dataset) may be differentiated, any descriptor may work even in a ML/DL model. Yet, if such a descriptor carries little chemical intuition or information, training with the descriptors are most likely to be difficult, requiring sophisticated models (and chemical rules), as well as a large amount of data to approximate the underlying one-to-one relationship between the descriptor and the property of interest. Conversely, when a molecular representation not only differentiates molecules but also bears rich chemical information such as those used in various embodiments of the invention, the training is straightforward even with a small set of data. In addition, the inventors are considerably expanded their studies by utilizing electron-density iso-surfaces of single molecules in the Solubility Challenges, fully optimized or kept of the same conformers when generating Hirshfeld surfaces. In further embodiments, molecules in a much larger dataset, ESOL (1128 molecules), are evaluated which is widely used by benchmarking efforts of machine and deep learning.

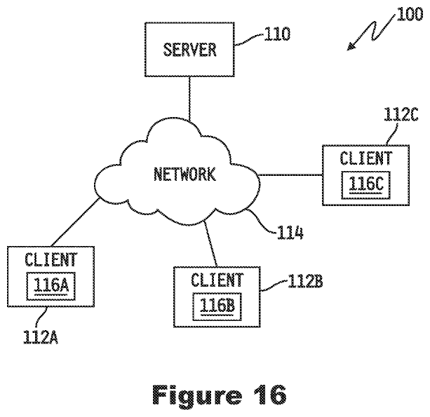

FIG. 16 is a high-level block diagram of a computing environment 100 according to one embodiment, although those skilled in the art of computing recognize that the components of the processing described herein may be conducted on a single computing machine or distributed over wired or wireless networks. FIG. 1 illustrates server 110 and three clients 112 connected by network 114. Only three clients 112 are shown in FIG. 1 in order to simplify and clarify the description. Embodiments of the computing environment 100 may have thousands or millions of clients 112 connected to network 114, for example the Internet. Users (not shown) may operate software 116 on one of clients 112 to both send and receive messages network 114 via server 110 and its associated communications equipment and software (not shown).

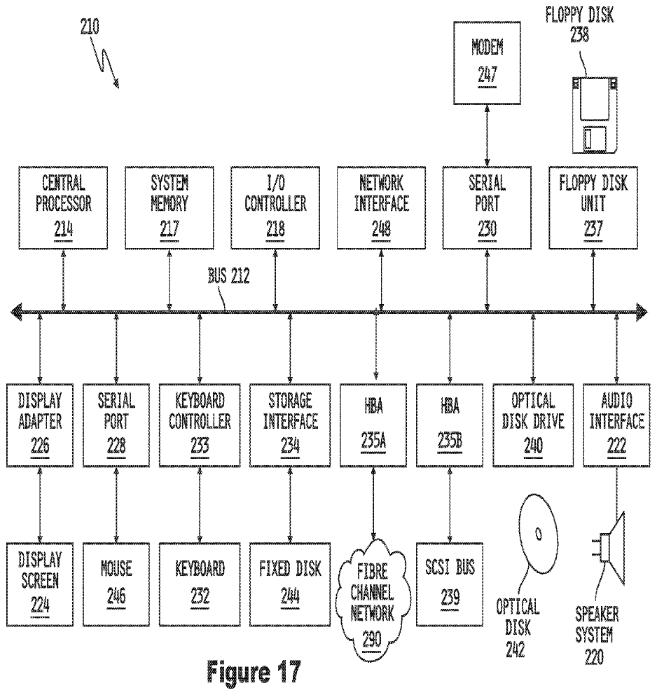

FIG. 17 depicts a block diagram of computer system 210 suitable for implementing server 110 or client 112. Computer system 210 includes bus 212 which interconnects major subsystems of computer system 210, such as central processor 214, system memory 217 (typically RAM, but which may also include ROM, flash RAM, or the like), input/output controller 218, external audio device, such as speaker system 220 via audio output interface 222, external device, such as display screen 224 via display adapter 226, serial ports 228 and 230, keyboard 232 (interfaced with keyboard controller 233), storage interface 234, disk drive 237 operative to receive floppy disk 238, host bus adapter (HBA) interface card 235A operative to connect with Fibre Channel network 290, host bus adapter (HBA) interface card 235B operative to connect to SCSI bus 239, and optical disk drive 240 operative to receive optical disk 242. Also included are mouse 246 (or other point-and-click device, coupled to bus 212 via serial port 228), modem 247 (coupled to bus 212 via serial port 230), and network interface 248 (coupled directly to bus 212).

Bus 212 allows data communication between central processor 214 and system memory 217, which may include read-only memory (ROM) or flash memory (neither shown), and random access memory (RAM) (not shown), as previously noted. RAM is generally the main memory into which operating system and application programs are loaded. ROM or flash memory may contain, among other software code, Basic Input-Output system (BIOS) which controls basic hardware operation such as interaction with peripheral components. Applications resident with computer system 210 are generally stored on and accessed via computer readable media, such as hard disk drives (e.g., fixed disk 244), optical drives (e.g., optical drive 240), floppy disk unit 237, or other storage medium (disk drive 237 is used to represent various type of removable memory such as flash drives, memory sticks and the like). Additionally, applications may be in the form of electronic signals modulated in accordance with the application and data communication technology when accessed via network modem 247 or interface 248 or other telecommunications equipment (not shown).

Storage interface 234, as with other storage interfaces of computer system 210, may connect to standard computer readable media for storage and/or retrieval of information, such as fixed disk drive 244. Fixed disk drive 244 may be part of computer system 210 or may be separate and accessed through other interface systems. Modem 247 may provide direct connection to remote servers via telephone link or the Internet via an internet service provider (ISP) (not shown). Network interface 248 may provide direct connection to remote servers via direct network link to the Internet via a POP (point of presence). Network interface 248 may provide such connection using wireless techniques, including digital cellular telephone connection, Cellular Digital Packet Data (CDPD) connection, digital satellite data connection or the like.

Many other devices or subsystems (not shown) may be connected in a similar manner (e.g., document scanners, digital cameras and so on). Conversely, all of the devices shown in FIG. 2 need not be present to practice the present disclosure. Devices and subsystems may be interconnected in different ways from that shown in FIG. 2. Operation of a computer system such as that shown in FIG. 2 is readily known in the art and is not discussed in detail in this application. Software source and/or object codes to implement the present disclosure may be stored in computer-readable storage media such as one or more of system memory 217, fixed disk 244, optical disk 242, or floppy disk 238. The operating system provided on computer system 210 may be a variety or version of either MS-DOS® (MS-DOS is a registered trademark of Microsoft Corporation of Redmond, Washington), WINDOWS® (WINDOWS is a registered trademark of Microsoft Corporation of Redmond, Washington), OS/2® (OS/2 is a registered trademark of International Business Machines Corporation of Armonk, New York), UNIX® (UNIX is a registered trademark of X/Open Company Limited of Reading, United Kingdom), Linux® (Linux is a registered trademark of Linus Torvalds of Portland, Oregon), or other known or developed operating system. In some embodiments, computer system 210 may take the form of a tablet computer, typically in the form of a large display screen operated by touching the screen. In tablet computer alternative embodiments, the operating system may be iOS® (iOS is a registered trademark of Cisco Systems, Inc. of San Jose, California, used under license by Apple Corporation of Cupertino, California), Android® (Android is a trademark of Google Inc. of Mountain View, California), Blackberry® Tablet OS (Blackberry is a registered trademark of Research In Motion of Waterloo, Ontario, Canada), webOS (webOS is a trademark of Hewlett-Packard Development Company, L.P. of Texas), and/or other suitable tablet operating systems.

Moreover, regarding the signals described herein, those skilled in the art recognize that a signal may be directly transmitted from a first block to a second block, or a signal may be modified (e.g., amplified, attenuated, delayed, latched, buffered, inverted, filtered, or otherwise modified) between blocks. Although the signals of the above described embodiments are characterized as transmitted from one block to the next, other embodiments of the present disclosure may include modified signals in place of such directly transmitted signals as long as the informational and/or functional aspect of the signal is transmitted between blocks. To some extent, a signal input at a second block may be conceptualized as a second signal derived from a first signal output from a first block due to physical limitations of the circuitry involved (e.g., there will inevitably be some attenuation and delay). Therefore, as used herein, a second signal derived from a first signal includes the first signal or any modifications to the first signal, whether due to circuit limitations or due to passage through other circuit elements which do not change the informational and/or final functional aspect of the first signal.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

### Appendix

| CATEGORY | PROPERTY | DATA SOURCE | NOTE |
|---|---|---|---|
| Drug | Water solubility | Literature | About 160 solubility values were collected from the well-curated |
| developability, | | | solubility challenge datasets.^{58, 59} |
| ADME, | Membrane permeability | ChEMBL | More than 3000 Caco-2 cell permeability values are available. |
| and toxicity | P-glycoprotein binding | ChEMBL | ID#4302 reports binding efficacies of 2,631 compounds. |
| | CYP450 binding | PubChem | 14,567 drug-like molecules were obtained from A1851. |
| | Microsomal clearance | ChEMBL | A curated dataset reports 5,348 human microsomal clearance |
| | | | values obtained from ChEMBL.⁶⁸ |
| | HEK293 cell toxicity | NCATS OpenData | 9789 entries are reported in the database. |
| | Human pregnane | PubChem/Tox21 | A1347033 has 9,666 compounds with 2,076 being agonists. Five |
| | X receptor (hPXR) | | confirmative assays are also available. |
| | hERG Inhibition | ChEMBL | ID#240 assay has more than 19,000 associated compounds. |
| | | | Several other databases are also available.^{70, 71} |
| | Drug-induced liver | FDA | The DILIrank database includes four classes (most, less likely, |
| | injury (DILD) | | no, indefinite concerning) of 1,036 FDA-approved drugs according |
| | | | to their potential for causing DILI.⁷² |
| Rare and | Malaria | PubChem | A1159554 and A1159566 report 384 active compounds out of |
| infectious | | | more than 94,000 being screened. |
| diseases | Ebola | Literature | Cell-based assays of 1,012 FDA-approved drugs against Ebola |
| | | | (and a few other viruses) were reported.⁷³ |
| | COVID-19 | NCATS OpenData | Several curated in vitro assay datasets including more than |
| | | | 10,000 entres of the cytopathic effect against SARS-CoV-2 |
| | | | receptor (ACE2) expressed in Vero E6 cells are available. |
| | | PubChem | A1706 reports 405 active inhibitors of SARS coronavirus 3C-like |
| | | | Protease (3CLPro) by screening more than 290,000 molecules. |
| | HIV | PubChem | A179 inhibiting assay dataset against HIV1 infected human CEM |
| | | | cells. It reports 1,812 active compounds out of more than 40,000. |
| Substance | Oxytocin receptor | PubChem | A2435 reports 1,044 active agonists out of more than 324,000 |
| abuse protein | (OXTR) | | compounds by cell-based high throughput screening assay. |
| targets | Opioid receptors | PubChem | A504326 reports 993 active agonists out of more than 335,000 |
| | | | compounds by cell-based high throughput screening of |
| | | | heterodimerization of the mu 1 (OPRM1) and delta 1 (OPRD1) receptors |

What is claimed is:

1. A method for creating a representation of a molecule as a chemically authentic and dimensionally reduced feature for computing molecular interactions and pertinent properties, the method comprising the steps of:
measuring a molecule by observation of electronic patterns on a molecular surface;
creating a manifold embedding of the observed electronic patterns;
associating the manifold embedding with chemical properties; and
creating a data structure based on the molecular surface and the chemical properties.

2. The method of claim 1 wherein the creating a data structure step includes creating a two-dimensional data structure.

3. The method of claim 2 wherein the manifold embedding step involves enclosing the molecular surface and excluding a portion of the surface points from the two-dimensional data structure.

4. The method of claim 3 wherein the excluding includes manifold cutting.

5. The method of claim 4 wherein the excluding includes manifold cutting between two surface points intercepted by principal axes of mesh points.

6. The method of claim 5 wherein the step of creating a structure involves linearly combining embedded observed electrical patterns.

7. The method of claim 1 further comprising a plurality of measuring, creating a manifold, associating, and creating a data structure steps to create a database of data structures representing a plurality of molecules.

8. The method of claim 7 further comprising determining whether a first data structure represents a conformer to a second data structure, wherein the first and second data structures representing respective conformers are stacked in the database.

9. A database of molecular representations, a plurality of data structures of the database comprising:
manifold embedding of the observed electronic patterns represented in two-dimensions based on the observed molecular surface;
an association of the manifold embedding with chemical properties.

10. A method of determining chemical properties of an observed molecule electronic patterns using a database having a plurality of data structures representing molecular surfaces and associated chemical properties, the method comprising the steps of:
creating a quantum calculation of the observed molecule electronic patterns;
creating a MEMS representation of the observed molecule using dimensionality reduction;
using the MEMS representation of the observed molecule with a neural network utilizing the database to identify properties of the observed molecule.

11. The method of claim 10 wherein the step of creating a MEMS representation includes performing GPLVM or VEA.

12. The method of claim 10 wherein the step of creating a MEMS representation includes identifying features of the observed molecule and mapping the features to a latent space.

13. The method of claim 10 wherein the step of creating a quantum calculation includes creating a manifold embedding of the observed electronic patterns.

14. The method of claim 13 wherein the maniform embedding step includes enclosing the molecular surface and excluding a portion of the surface points from the step of creating a MEMS representation.

15. The method of claim 14 wherein the excluding includes manifold cutting.

16. The method of claim 15 wherein the manifold cutting includes cutting between two surface points intercepted by principal axes of mesh points.

17. A method of designing molecules for particular bioavailable properties, the method comprising the steps of:
creating a latent space of molecular projections on a functional surface; and
transforming a feature matrix based on the latent space to an adjacency matrix matching molecular projections with functional properties.

18. The method of claim 17 wherein the step of creating a latent space includes creating MEMS projections by GPLVM or VAE, performing Bayesian optimization to map the MEMS projection on the functional surface.

19. The method of claim 17 wherein the step of transforming includes creating a plurality of deepsets from the feature matrix, performing machine learning on the plurality of deepsets, and creating the adjacency matrix.

20. A database formed by any of the preceding claims.
